(19) United States
(12) Patent Application Publication
(10) Pub. No.: US 2026/0115792 A1
(43) Pub. Date: Apr. 30, 2026

(54) METHOD FOR FORMING A TUBE

(71) Applicant: MiRus LLC, Marietta, GA (US)

(72) Inventor: Noah Roth, Marietta, GA (US)

(21) Appl. No.: 19/375,452

(22) Filed: Oct. 31, 2025

Related U.S. Application Data
(63) Continuation-in-part of application No. 18/577,610.
(60) Provisional application No. 63/226,289, filed on Jul. 28, 2021.

(51) Int. Cl. B22F 3/16; B22F 3/24; B22F 5/10

(52) U.S. Cl. CPC B22F 3/162; B22F 3/24; B22F 5/10; B22F 2003/248; B22F 2201/01; B22F 2301/35

(57) ABSTRACT

A medical device that is at least partially formed of a metal material and a method for forming the medical device.

### REFERENCED APPLICATIONS

The present application is a continuation-in-part of U.S. patent application Ser. No. 18/577,610 filed Jan. 8, 2024, which is a nationalized application of PCT Application Serial No. PCT/US2022/038629 filed Jul. 28, 2022 (published as WO 2023/009695) (now expired), which in turn claims priority on U.S. Provisional Application Ser. No. 63/226,289 filed Jul. 28, 2021 (now expired), all of which are incorporated herein by reference.

### FIELD OF DISCLOSURE

The disclosure relates generally to medical devices and medical device applications, and particularly to metal rods and metal tubes that are used to form all or part of a medical device.

### SUMMARY OF THE DISCLOSURE

The present disclosure is direct to a medical device that is partially or fully formed of a metal alloy. As can be appreciated, the medical device can optionally be formed of other materials (e.g., material containing one or more carbon fibers, boron fibers glass fibers, aramid fibers [Kevlar, Twaron, etc.], basalt fibers, animal tissue, polymers, textiles, etc.).

In one non-limiting aspect of the present disclosure, the metal alloy is used to at least partially form the medical device has one or more improved properties (e.g., (e.g., strength, durability, hardness, biostability, bendability, coefficient of friction, radial strength, flexibility, tensile strength, tensile elongation, stress-strain properties, reduced recoil, radiopacity, heat sensitivity, biocompatibility, improved fatigue life, crack resistance, crack propagation resistance, reduced magnetic susceptibility, etc.), improved conformity when bent, less recoil, increase yield strength, improved fatigue ductility, improved durability, improved fatigue life, reduced adverse tissue reactions, reduced metal ion release, reduced corrosion, reduced allergic reaction, improved hydrophilicity, reduced toxicity, reduced thickness of metal component, improved bone fusion, and/or lower ion release into tissue, etc.) of such medical device. These one or more improved physical properties of the metal alloy can be achieved in the medical device without having to increase the bulk, volume and/or weight of the medical device, and in some instances these improved physical properties can be obtained even when the volume, bulk and/or weight of the medical device is reduced as compared to medical devices that are at least partially formed from traditional stainless-steel or cobalt and chromium alloy materials. However, it will be appreciated that the metal alloy can include metals such as stainless-steel, cobalt and chromium, etc.

The metal alloy that is used to at least partially form the medical device 1) increase the radiopacity of the medical device, 2) increase the radial strength of the medical device, 3) increase the yield strength and/or ultimate tensile strength of the medical device, 4) improve the stress-strain properties of the medical device, 5) improve the crimping and/or expansion properties of the medical device, 6) improve the bendability and/or flexibility of the medical device, 7) improve the strength and/or durability of the medical device, 8) increase the hardness of the medical device, 9) improve the recoil properties of the medical device, 10) improve the biostability and/or biocompatibility properties of the medical device, 11) increase fatigue resistance of the medical device, 12) resist cracking in the medical device and resist propagation of cracks, 13) enable smaller, thinner, and/or lighter weight medical device to be made, 14) reduce the outer diameter of a crimped medical device, 15) improve the conformity of the medical device to the shape of the treatment area when the medical device is used and/or expanded in the treatment area, 16) reduce the amount of recoil of the medical device to the shape of the treatment area when the medical device is expanded in the treatment area, 17) increase yield strength of the medical device, 18) improve fatigue ductility of the medical device, 18) improve durability of the medical device, 19) improve fatigue life of the medical device, 20) reduce adverse tissue reactions after implant of the medical device, 21) reduce metal ion release after implant of the medical device, 22) reduce corrosion of the medical device after implant of the medical device, 23) reduce allergic reaction after implant of the medical device, 24) improve hydrophilicity of the medical device, 25) reduce thickness of meta component of medical device, 26) improve bone fusion with medical device, 27) lower ion release from medical device into tissue, 28) reduce magnetic susceptibility of the medical device when implanted in a patient, and/or 29) reduce toxicity of the medical device after implant of the medical device. The metal alloy generally includes one or more materials that impart the desired properties to the medical device so as to withstand the manufacturing processes that are needed to produce the medical device. These manufacturing processes can include, but are not limited to, laser cutting, etching, crimping, annealing, drawing, pilgering, electroplating, electro-polishing, machining, plasma coating, 3D printing, 3D printed coatings, chemical vapor deposition, chemical polishing, cleaning, pickling, ion beam deposition or implantation, sputter coating, vacuum deposition, etc. In one non-limiting embodiment, the medical device is partially or fully formed by a 3D printing process.

In another non-limiting aspect of the present disclosure, a medical device that can include the metal alloy can be an orthopedic device; PFO (patent foramen ovale) device; stent; valve (e.g., heart valve, TAVR valve, etc.); valve frame; expandable frame of a medical device, spinal implant; frame and other structures for use with a spinal implant; vascular implant; graft; guide wire; sheath; catheter; needle; stent catheter; electrophysiology catheter; hypotube; staple; cutting device; any type of implant; pacemaker; dental implant; dental crown; dental braces; wire for used in medical procedures; bone implant; prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage; nail; rod; screw; post; cage; plate; pedicle screw; cap; hinge; joint system; anchor; spacer; shaft; anchor; disk; ball; tension band; locking connector other structural assembly that is used in a body to support a structure, mount a structure and/or repair a structure in a body such as, but not limited to, a human body. In one non-limiting application, the medical device is a spinal implant. In another non-limiting application, the medical device is a prosthetic device. In another non-limiting application, the medical device is a stent. In another non-limiting application, the medical device is a TAVR valve.

Although the present disclosure will be described with particular reference to medical devices, it will be appreciated that the metal alloy can be used in other components that are subjected to stresses that can lead to cracking and fatigue failure (e.g., automotive parts, springs, aerospace parts, industrial machinery and parts, tools (e.g., medical tools, industrial tools, household tools), etc.).

In accordance with another and/or alternative non-limiting aspect of the present disclosure, the medical device can be formed of a variety of materials. In one non-limiting embodiment, the medical device is partially (e.g. 1-99.999 wt. % and all values and ranges therebetween) or fully formed of a metal alloy that includes a) stainless-steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, m) metal alloy (e.g., stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween), or n) metal alloy (e.g., stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) hafnium (e.g., 5-99 awt. % hafnium and all values and ranges therebetween). As used herein, atomic weight percent (awt. %) or atomic percentage (awt %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of rhenium and/or hafnium results in the ductility and/or tensile strength of the metal alloy to improve as compared to a metal alloy is that absent rhenium and/or hafnium. Such improvement in ductility and/or tensile strength due to the inclusion of at least 5-15 awt. % rhenium and/or 5-15 awt. % hafnium in the metal alloy is referred to as the “rhenium effect” or “hafnium effect.” As defined herein, a “rhenium effect” or a “hafnium effect” is a) an increase of at least 10% in ductility of the metal alloy caused by the addition of rhenium and/or hafnium to the metal alloy, and/or b) an increase of at least 10% in tensile strength of the metal alloy caused by the addition of rhenium and/or hafnium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative aspect of the present disclosure, the metal alloy used to partially or fully form the medical device includes stainless-steel, CoCr alloys, TiAlV alloys, aluminum alloys, nickel alloys, titanium alloys, tungsten alloys, molybdenum alloys, copper alloys, MP35N alloys, or beryllium-copper alloys that have been modified to include at least 5-15 awt. % rhenium and/or 5-15 awt % hafnium so as to result in improved ductility and/or tensile strength as compared to the same metal alloy that is absent rhenium and/or hafnium. As defined herein, a stainless-steel alloy (SS alloy) includes at least 50 wt. % iron (e.g., 50-85 wt. % and all values and ranges therebetween), 10-30 wt. % chromium, 0-35 wt. % nickel, and optionally one or more of 0-5 wt. % molybdenum, 0-6 wt. % manganese, 0-1 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316 L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-72 wt. % cobalt, 15-35 wt. % chromium, and optionally one or more of 1-38 wt. % nickel, 2-18 wt. % molybdenum, 0-18 wt. % iron, 0-1 wt. % titanium, 0-2.8 wt. % manganese, 0-0.15 wt. % silver, 0-2 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox and Elgiloy alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % ocadmium, 0-0.05 wt. % obismuth, 0-1 wt. % nickel, 0-0.2 wt. % ovanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium.

In accordance with another and/or alternative aspect of the present disclosure, the metal alloy used to partially or fully form the medical device includes at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a rhenium effect. In one non-limiting embodiment, the metal alloy is a stainless-steel alloy that has been modified to include at least 5-15 awt. % rhenium and/or hafnium. In another and/or alternative non-limiting embodiment, the metal alloy is a cobalt-chromium alloy that has been modified to include at least 5-15 awt. % rhenium and/or hafnium. In another and/or alternative non-limiting embodiment, the metal alloy is a TiAlV alloy that has been modified to include at least 5-15 awt. % rhenium and/or hafnium. In another and/or alternative non-limiting embodiment, the metal alloy is an aluminum alloy that has been modified to include at least 5-15 awt. % rhenium and/or hafnium. In another and/or alternative non-limiting embodiment, the metal alloy is a nickel alloy that has been modified to include at least 5-15 awt. % rhenium and/or hafnium. In another and/or alternative non-limiting embodiment, the metal alloy is a titanium alloy that has been modified to include at least 5-15 awt. % rhenium and/or hafnium. In another and/or alternative non-limiting embodiment, the metal alloy is a tungsten alloy that has been modified to include at least 15 awt. % rhenium and/or hafnium. In another and/or alternative non-limiting embodiment, the metal alloy is a molybdenum alloy that has been modified to include at least 5-15 awt. % rhenium and/or hafnium. In another and/or alternative non-limiting embodiment, the metal alloy is a copper alloy that has been modified to include at least 5-15 awt. % rhenium and/or hafnium. In another and/or alternative non-limiting embodiment, the metal alloy is a beryllium-copper alloy that has been modified to include at least 5-15 awt. % rhenium and/or hafnium.

In accordance with another and/or alternative aspect of the present disclosure, the metal alloy used to partially or fully form the medical device includes at least 5 awt. % (e.g., 5-99 awt. % and all values and ranges therebetween) hafnium, and 0.1-96 wt. % (and all values and ranges therebetween) of one or more additives selected from the group of aluminum, boron, beryllium, bismuth, cadmium, calcium, cerium, chromium, cobalt, copper, gallium, gold, iridium, iron, lanthanum, lithium, magnesium, manganese, molybdenum, nickel, niobium, osmium, palladium, platinum, rare earth metals, rhenium, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a hafnium effect. In one non-limiting embodiment, the metal alloy is a stainless-steel alloy that has been modified to include at least 5-15 awt. % hafnium. In another and/or alternative non-limiting embodiment, the metal alloy is a cobalt chromium alloy that has been modified to include at least 5-15 awt. % hafnium. In another and/or alternative non-limiting embodiment, the metal alloy is a TiAlV alloy that has been modified to include at least 5-15 awt. % hafnium. In another and/or alternative non-limiting embodiment, the metal alloy is an aluminum alloy that has been modified to include at least 5-15 awt. % hafnium. In another and/or alternative non-limiting embodiment, the metal alloy is a nickel alloy that has been modified to include at least 5-15 awt. % hafnium. In another and/or alternative non-limiting embodiment, the metal alloy is a titanium alloy that has been modified to include at least 5-15 awt. % hafnium. In another and/or alternative non-limiting embodiment, the metal alloy is a tungsten alloy that has been modified to include at least 5-15 awt. % hafnium. In another and/or alternative non-limiting embodiment, the metal alloy is a molybdenum alloy that has been modified to include at least 5-15 awt. % hafnium. In another and/or alternative non-limiting embodiment, the metal alloy is a copper alloy that has been modified to include at least 5-15 awt. % hafnium. In another and/or alternative non-limiting embodiment, the metal alloy is a beryllium-copper alloy that has been modified to include at least 5-15 awt. % hafnium.

In accordance with another and/or alternative aspect of the present disclosure, the metal alloy used to partially or fully form the medical device includes rhenium and/or hafnium, and molybdenum, and the weight percent of rhenium and/or hafnium in the metal alloy is optionally greater than the weight percent of molybdenum in the metal alloy, and the weight percent of one or more additive (e.g., aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium) in the metal alloy is optionally greater that the weight percent of molybdenum in the metal alloy, and the metal alloy optionally includes 0-2 wt. % of a combination of other components other than the additives (e.g., carbon, oxygen, phosphorous, sulfur, hydrogen, lead, nitrogen, etc.). In one non-limiting embodiment, the metal alloy is fully formed of or includes rhenium and/or hafnium, and molybdenum, and the weight percent of rhenium and/or hafnium plus the combined weight percent of additives is greater than the weight percent of molybdenum, and the metal alloy optionally includes 0-2 wt. % of a combination of other components other than the additives (e.g., carbon, oxygen, phosphorous, sulfur, hydrogen, lead, nitrogen, etc.).

In accordance with another and/or alternative aspect of the present disclosure, the metal alloy used to partially or fully form the medical device includes rhenium and/or hafnium, and molybdenum, and one or more additives selected from bismuth, niobium, tantalum, tungsten, titanium, vanadium, chromium, manganese, yttrium, zirconium, technetium, ruthenium, rhodium, hafnium, osmium, copper, and iridium, and the atomic weight percent of rhenium and/or hafnium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal alloy used to partially or fully form the medical device includes at least 5 awt. % (e.g., 5-99 awt. % and all values and ranges therebetween) rhenium and/or hafnium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another and/or alternative non-limiting embodiment, the metal alloy includes rhenium and/or hafnium, molybdenum, and chromium. In another and/or alternative non-limiting embodiment, the metal alloy includes at least 35 wt. % (e.g., 35-75 wt. % and all values and ranges therebetween) rhenium and/or hafnium, and the metal alloy also includes chromium. In one non-limiting embodiment, the metal alloy includes at least 35 wt. % rhenium and/or hafnium, and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen. In another and/or alternative non-limiting embodiment, the metal alloy includes 15-50 awt. % rhenium and/or hafnium (and all values and ranges therebetween) and 0.5-70 awt. % chromium (and all values and ranges therebetween). In another and/or alternative non-limiting embodiment, the metal alloy includes 15-50 awt. % rhenium and/or hafnium (and all values and ranges therebetween) and 0.5-70 awt. % tantalum (and all values and ranges therebetween). In another and/or alternative non-limiting embodiment, the metal alloy includes 15-50 awt. % rhenium and/or hafnium (and all values and ranges therebetween) and 0.5-70 awt. % niobium (and all values and ranges therebetween). In another and/or alternative non-limiting embodiment, the metal alloy includes 15-50 awt. % rhenium and/or hafnium (and all values and ranges therebetween) and 0.5-70 awt. % titanium (and all values and ranges therebetween). In another and/or alternative non-limiting embodiment, the metal alloy includes 15-50 awt. % rhenium and/or hafnium (and all values and ranges therebetween) and 0.5-70 awt. % zirconium (and all values and ranges therebetween). In another and/or alternative non-limiting embodiment, the metal alloy includes 15-50 awt. % rhenium and/or hafnium (and all values and ranges therebetween) and 0.5-70 awt. % molybdenum (and all values and ranges therebetween). In another and/or alternative non-limiting embodiment, the metal alloy includes at least 15 awt. % rhenium and/or hafnium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In accordance with another and/or alternative aspect of the present disclosure, the metal alloy used to partially or fully form the medical device includes a refractory metal alloy, and wherein the refractory metal alloy includes at least 20 wt. % of one or more of niobium, rhenium, tantalum, molybdenum or tungsten (e.g., 20-99.9 wt. % and all values and ranges therebetween), and 0.1-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the medical device includes a refractory metal alloy, and wherein the refractory metal alloy includes at least 5 awt. % rhenium and/or hafnium, at least 20 wt. % of one or more of niobium, tantalum, molybdenum or tungsten (e.g., 20-99.9 wt. % and all values and ranges therebetween), and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the medical device includes a refractory metal alloy, and wherein the refractory metal alloy includes at least 20 wt. % of one or more of niobium, tantalum or tungsten, and wherein the refractory metal alloy includes 0-30 wt. % molybdenum (and all values and ranges therebetween), and wherein the refractory metal alloy includes at least 5 awt. % rhenium and/or hafnium (e.g., 5-80 awt. % rhenium and/or hafnium and all values and ranges therebetween), and wherein the refractory metal alloy includes and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the medical device includes at least 5 awt. % rhenium and/or hafnium (e.g., 5-99 awt. % rhenium and/or hafnium and all values and ranges therebetween), and at least 0.1 wt. % of one or more additive metals selected from aluminum, bismuth, chromium, cobalt, copper, hafnium, iridium, iron, magnesium, manganese, nickel, niobium, osmium, rhodium, ruthenium, silicon, silver, tantalum, technetium, titanium, tungsten, vanadium, yttrium, and zirconium, and wherein the metal alloy includes 0-30 wt. % molybdenum (and all values and ranges therebetween), and wherein a combined weight percent of rhenium and/or hafnium, and the additive metals is 70-100 wt. % (and all values and ranges therebetween).

In accordance with another and/or alternative aspect of the present disclosure, the metal alloy used to partially or fully form the medical device includes stainless-steel that has been modified with at least 5 awt. % rhenium and/or hafnium (e.g., 5-50 awt. % rhenium and/or hafnium and all values and ranges therebetween), and wherein a combined weight percent of iron, chromium, nickel, tantalum, niobium, copper, manganese, aluminum, titanium, selenium, vanadium, tungsten, hafnium and rhenium is 70-100 wt. % (and all values and ranges therebetween).

In accordance with another and/or alternative aspect of the present disclosure, the metal alloy used to partially or fully form the medical device includes cobalt-chromium alloy that has been modified with at least 5 awt. % rhenium and/or hafnium (e.g., 5-50 awt. % rhenium and/or hafnium and all values and ranges therebetween), and wherein a combined weight percent of cobalt, chromium, nickel, iron, titanium, manganese, silver, tungsten, silicon, aluminum, iron, boron, silver, titanium, hafnium, and rhenium is 70-100 wt. % (and all values and ranges therebetween).

In accordance with another and/or alternative aspect of the present disclosure, the metal alloy used to partially or fully form the medical device includes titanium-aluminum-vanadium alloy that has been modified with at least 5 awt. % rhenium and/or hafnium (e.g., 5-50 awt. % rhenium and/or hafnium and all values and ranges therebetween), and wherein a combined weight percent of aluminum, vanadium, titanium, iron, yttrium, hafnium, and rhenium is 70-100 wt. % (and all values and ranges therebetween).

In accordance with another and/or alternative aspect of the present disclosure, the metal alloy used to partially or fully form the medical device includes aluminum alloy that has been modified with at least 5 awt. % rhenium and/or hafnium (e.g., 5-50 awt. % rhenium and/or hafnium and all values and ranges therebetween), and wherein a combined weight percent of aluminum, silicon, magnesium, manganese, scandium, beryllium, yttrium, cerium, chromium, iron, zinc, titanium, lithium, silver, calcium, zirconium, cadmium, bismuth, nickel, vanadium, gallium, copper, hafnium, and rhenium is 70-100 wt. % (and all values and ranges therebetween).

In accordance with another and/or alternative aspect of the present disclosure, the metal alloy used to partially or fully form the medical device includes nickel alloy that has been modified with at least 5 awt. % rhenium and/or hafnium (e.g., 5-50 awt. % rhenium and/or hafnium and all values and ranges therebetween), and wherein a combined weight percent of nickel, chromium, iron, copper, cobalt, aluminum, tantalum, tungsten, titanium, niobium, silicon, hafnium, and rhenium is 70-100 wt. % (and all values and ranges therebetween).

In accordance with another and/or alternative aspect of the present disclosure, the metal alloy used to partially or fully form the medical device includes titanium alloy that has been modified with at least 5 awt. % rhenium and/or hafnium (e.g., 5-50 awt. % rhenium and/or hafnium and all values and ranges therebetween), and wherein a combined weight percent of titanium, aluminum, tin, palladium, vanadium, nickel, ruthenium, chromium, zirconium, niobium, silicon, cobalt, iron, hafnium, and rhenium is 70-100 wt. % (and all values and ranges therebetween).

In accordance with another and/or alternative aspect of the present disclosure, the metal alloy used to partially or fully form the medical device includes tungsten alloy that has been modified with at least 5 awt. % rhenium and/or hafnium (e.g., 5-50 awt. % rhenium and/or hafnium and all values and ranges therebetween), and wherein a combined weight percent of tungsten, nickel, copper, iron, hafnium, and rhenium is 70-100 wt. % (and all values and ranges therebetween).

In accordance with another and/or alternative aspect of the present disclosure, the metal alloy used to partially or fully form the medical device includes copper alloy that has been modified with at least 5 awt. % rhenium and/or hafnium (e.g., 5-50 awt. % rhenium and/or hafnium and all values and ranges therebetween), and wherein a combined weight percent of copper, zinc, tin, iron, silicon, manganese, aluminum, beryllium, cobalt, nickel, hafnium, and rhenium is 70-100 wt. % (and all values and ranges therebetween).

In accordance with another and/or alternative aspect of the present disclosure, the metal alloy used to partially or fully form the medical device includes beryllium-copper alloy that has been modified with at least 5 awt. % rhenium and/or hafnium (e.g., 5-50 awt. % rhenium and/or hafnium and all values and ranges therebetween), and wherein a combined weight percent of copper, beryllium, cobalt, silicon, hafnium, and rhenium is 70-100 wt. % (and all values and ranges therebetween).

In accordance with another and/or alternative aspect of the present disclosure, the metal alloy used to partially or fully form the medical device includes titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In accordance with another and/or alternative aspect of the present disclosure, the metal alloy used to partially or fully form the medical device includes a metal alloy that includes less than 5 wt. % nickel (e.g., 0-4.99 wt. % and all values and ranges therebetween).

In accordance with another and/or alternative aspect of the present disclosure, the metal alloy used to partially or fully form the medical device includes a metal alloy that includes less than 5 wt. % chromium (e.g., 0-4.99 wt. % and all values and ranges therebetween).

In accordance with another and/or alternative aspect of the present disclosure, the metal alloy used to partially or fully form the medical device includes a primary metal (5-95 wt. % primary metal and all values and ranges therebetween) selected from one or more of molybdenum, rhenium, hafnium, niobium, tantalum, tungsten, and one or more alloying agents such as, but are not limited to, calcium, carbon, cerium oxide, chromium, cobalt, copper, gold, hafnium, iron, lanthanum oxide, magnesium, nickel, osmium, platinum, rare earth metals, rhenium, silver, technetium, titanium, vanadium, yttrium, yttrium oxide, zinc, zirconium, zirconium oxide, and/or alloys of one or more of such components (e.g., MoHfC, MoY₂O₃, MoCs₂O, MoW, MoTa, MoZrO₂, MoLa₂O₃, MoRe alloy, MoReW alloy, HfMo alloy, HfW alloy, ReW alloy, etc.).

In accordance with another and/or alternative aspect of the present disclosure, the metal alloy used to partially or fully form the medical device includes tungsten and copper and optionally one or more metal agents such as, but are not limited to, calcium, carbon, cerium oxide, chromium, cobalt, gold, hafnium, iron, lanthanum oxide, magnesium, molybdenum, nickel, niobium, osmium, platinum, rare earth metals, rhenium, silver, tantalum, technetium, titanium, vanadium, yttrium, yttrium oxide, zinc, zirconium, zirconium oxide, and/or alloys of one or more of such components. In one non-limiting formulation, the metal alloy includes 1-99.9 wt. % tungsten (and all values and ranges therebetween) (e.g., 1 wt. %, 1.01 wt. %, 1.02 wt. % . . . 99.88 wt. %, 99.89 wt. %, 99.9 wt. %), and 0.1-99 wt. % copper (and all values and ranges therebetween) (e.g., 0.1 wt. %, 0.101 wt. %, 0.102 wt. % . . . 98.998 wt. %, 98.999 wt. %, 99 wt. %). In another non-limiting formulation, the tungsten constitutes the greatest weight percent in the metal alloy and the copper constitutes the second greatest weight percent in the metal alloy. In another non-limiting formulation, the tungsten constitutes the largest weight percent of any component that forms the metal alloy. In another non-limiting formulation, the tungsten constitutes greater than 50 wt. % of the metal alloy.

In accordance with another and/or alternative aspect of the present disclosure, the metal alloy used to partially or fully form the medical device includes tungsten and rhenium and/or hafnium, and optionally one or more alloying agents such as, but not limited to, calcium, carbon, cerium oxide, chromium, cobalt, copper, gold, hafnium, iron, lanthanum oxide, magnesium, molybdenum, nickel, niobium, osmium, platinum, rare earth metals, rhenium, silver, tantalum, technetium, titanium, tungsten, vanadium, yttrium, yttrium oxide, zinc, zirconium, zirconium oxide, and/or alloys of one or more of such components (e.g., WRe, WReMo, WHf, WHfMo, WHfRe, WHfReMo, etc.). In one non-limiting formulation, the metal alloy includes 1-40 wt. % rhenium and/or hafnium (and all values and ranges therebetween and 60-99 wt. % tungsten (and all values and ranges therebetween). The total weight percent of the tungsten, rhenium and hafnium in the metal alloy is at least about 95 wt. % (e.g., 95-100% and all values and ranges therebetween). In another non-limiting formulation, the metal alloy includes 1-47.5 wt. % rhenium and/or hafnium (and all values and ranges therebetween) and 20-80 wt. % tungsten (and all values and ranges therebetween) and 0-47.5 wt. % molybdenum (and all values and ranges therebetween). The total weight percent of the tungsten, molybdenum, rhenium and hafnium in the metal alloy is at least about 95 wt. % (e.g., 95-100% and all values and ranges therebetween). In one non-limiting specific metal alloy, the weight percent of the tungsten is greater than a weight percent of rhenium, hafnium and/or molybdenum. In another non-limiting specific metal alloy, the weight percent of the tungsten is greater than 50 wt. % of the metal alloy. In another non-limiting specific metal alloy, the weight percent of the tungsten is greater than a weight percent of rhenium and/or hafnium, but is less than a weigh percent of molybdenum. In another non-limiting specific metal alloy, the weight percent of the tungsten is greater than a weight percent of molybdenum, but less than a weigh percent of rhenium and/or hafnium. In another non-limiting metal alloy, the weight percent of the tungsten is less than a weight percent of rhenium, hafnium and/or molybdenum.

[In accordance with another and/or alternative aspect of the present disclosure, the metal alloy used to partially or fully form the medical device includes a metal alloy that has an atomic weight percent of rhenium and/or hafnium to the atomic weight percent of the combination of bismuth, niobium, tantalum, tungsten, titanium, vanadium, chromium, manganese, yttrium, zirconium, technetium, ruthenium, rhodium, hafnium, osmium, copper, and iridium in the metal alloy is 0.7:1 to 1.5:1 (and all values and ranges therebetween).

In accordance with another and/or alternative aspect of the present disclosure, the metal alloy used to partially or fully form the medical device includes two of bismuth, niobium, tantalum, tungsten, titanium, vanadium, chromium, manganese, yttrium, zirconium, technetium, ruthenium, rhodium osmium, copper, and iridium, the atomic ratio of the two metals is 0.4:1 to 2.5:1 (and all values and ranges therebetween).

In accordance with another and/or alternative aspect of the present disclosure, the metal alloy used to partially or fully form the medical device includes a titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In accordance with another and/or alternative non-limiting aspect of the present disclosure, at least 10 wt. % (e.g., 10-95 wt. % and all values and ranges therebetween) of the metal alloy that includes at least 15 atw. % hafnium and/or rhenium and also includes one or more of molybdenum, niobium, tantalum, or tungsten.

In accordance with another and/or alternative non-limiting aspect of the present disclosure, there is provided a metal alloy that includes at least 15 atw. % hafnium and/or rhenium and 0.1-75 wt. % (and all values and ranges therebetween) of one or more of aluminum, bismuth, calcium, carbon, chromium, cobalt, copper, gold, iridium, iron, lanthanum, magnesium, manganese, molybdenum, nickel, niobium, osmium, platinum, rare earth metals, rhodium, ruthenium, silver, tantalum, technetium, titanium, tungsten, vanadium, yttrium, zinc, zirconium, and/or alloys of one or more of such components.

In another non-limiting aspect of the present disclosure, the metals used to form the metal alloy includes at least 15 atw. % hafnium and/or rhenium, nickel and tungsten and optionally one or more alloying agents such as, but not limited to, aluminum, bismuth, calcium, carbon, chromium, cobalt, copper, gold, iron, magnesium, molybdenum, niobium, osmium, platinum, rare earth metals, rhenium, silver, tantalum, technetium, titanium, vanadium, yttrium, zinc, zirconium, and/or alloys of one or more of such components (e.g., WNi, WNiMo, WNi.Re, etc.). In another non-limiting formulation, the metal alloy that includes at least 15 atw. % hafnium and/or rhenium includes 1-47.5 wt. % nickel (and all values and ranges therebetween) and 20-80 wt. % tungsten (and all values and ranges therebetween) and 1-47.5 wt. % (and all values and ranges therebetween) of one or more of aluminum, bismuth, calcium, carbon, chromium, cobalt, copper, gold, hafnium, iron, magnesium, molybdenum, nickel, niobium, osmium, platinum, rare earth metals, silver, tantalum, technetium, titanium, vanadium, yttrium, zinc, zirconium.

In accordance with another and/or alternative non-limiting aspect of the present disclosure, the metal alloy includes at least 15 atw. % hafnium and/or rhenium and nickel.

In accordance with another and/or alternative aspect of the present disclosure, the metal alloy includes at least 15 atw. % hafnium and/or rhenium includes (e.g., 15-99 awt. % and all values and ranges therebetween), optionally 5 awt. % (e.g., 5-99 awt. % and all values and ranges therebetween) molybdenum, optionally 5 awt. % (e.g., 5-99 awt. % and all values and ranges therebetween) niobium, optionally 5 awt. % (e.g., 5-99 awt. % and all values and ranges therebetween) tantalum, optionally 5 awt. % (e.g., 5-99 awt. % and all values and ranges therebetween) tungsten, and at least 0.1 wt. % (e.g., 0.1 wt. % to 96 wt. % and all values and ranges therebetween) of one or more of aluminum, boron, beryllium, bismuth, cadmium, calcium, cerium, chromium, cobalt, copper, gallium, gold, iridium, iron, lanthanum, lithium, magnesium, manganese, nickel, osmium, palladium, platinum, rare earth metals, rhodium, ruthenium, scandium, silver, silicon, technetium, tin, titanium, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen.

In accordance with another and/or alternative non-limiting aspect of the present disclosure, there is provided a medical device or other type of device partially or fully formed of a metal alloy that includes at least 15 atw. % hafnium. In one non-limiting embodiment, 50-100% (and all values and ranges therebetween) of the medical device or other type of device is formed of the metal alloy that includes at least 15 atw. % hafnium. In another non-limiting embodiment, at least 30 wt. % (e.g., 30-100 wt. % and all values and ranges therebetween) of the medical device or other type of device is formed of a metal alloy that includes at least 15 atw. % hafnium.

In accordance with another and/or alternative aspect of the present disclosure, the metal alloy used to partially or fully form the medical device includes less than 5 wt. % nickel (e.g., 0-4.99 wt. % and all values and ranges therebetween).

In accordance with another and/or alternative aspect of the present disclosure, the metal alloy used to partially or fully form the medical device includes less than 5 wt. % chromium (e.g., 0-4.99 wt. % and all values and ranges therebetween).

In accordance with another and/or alternative non-limiting aspect of the present disclosure, the metal alloy that that partially or fully forms the medical device is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printing, 3D printed coatings, chemical vapor deposition, chemical polishing, cleaning, pickling, ion beam deposition or implantation, sputter coating, vacuum deposition, EDM cutting, gun drilling, compression, sintering, compression process, consolidation process, etc.

In accordance with another and/or alternative non-limiting aspect of the present disclosure, the metal alloy that partially or fully forms the medical devise optionally has a generally uniform density throughout the metal alloy, and also results in the desired yield and ultimate tensile strengths of the metal alloy. In one non-limiting embodiment, the density of the metal alloy that includes at least 15 atw. % rhenium and/or hafnium is generally at least about 5 gm/cc (e.g., 5 gm/cc-21 gm/cc and all values and ranges therebetween; 10-20 gm/cc; etc.), and typically at least about 11-19 gm/cc.

In accordance with another and/or alternative non-limiting aspect of the present disclosure, the metal alloy optionally includes a certain amount of carbon and oxygen; however, this is not required. The carbon to oxygen atomic ratio can be as low as about 0.2:1 (e.g., 0.2:1 to 50:1 and all values and ranges therebetween).

In accordance with another and/or alternative non-limiting aspect of the present disclosure, the metal alloy optionally includes a controlled amount of nitrogen; however, this is not required. In one non-limiting formulation, the metal alloy includes less than about 0.001 wt. % nitrogen (e.g., 0 wt. % to 0.0009999 wt. % and all values and ranges therebetween). In one non-limiting formulation of the metal alloy, the atomic ratio of carbon to nitrogen ion the metal alloy is at least about 1.5:1 (e.g., 1.5:1 to 400:1 and all values and ranges therebetween). In another non-limiting formulation of the metal alloy, the atomic ratio of oxygen to nitrogen is at least about 1.2:1 (e.g., 1.2:1 to 150:1 and all value and ranges therebetween).

In another and/or alternative non-limiting aspect of the present disclosure, the metal alloy 1) optionally is not clad, metal sprayed, plated, and/or formed (e.g., cold worked, hot worked, etc.) onto another metal, 2) optionally does not have another metal or metal alloy metal sprayed, plated, clad, and/or formed onto the metal alloy, 3) optionally is clad, metal sprayed, plated and/or formed (e.g., cold worked, hot worked, etc.) onto another metal, or 4) optionally has another metal or metal alloy metal sprayed, plated, clad and/or formed onto the metal alloy.

In accordance with another and/or alternative non-limiting aspect of the present disclosure, the medical device or other device can optionally be at least partially or fully formed from a tube or rod of metal alloy, or is formed into a shape that is at least 80% of the final net shape of the medical device (e.g., formed by 3D printing, formed by compression and/or sintering of metal alloy powder, etc.). When the metal alloy is formed into a rod, the rod can optionally be gun drilled or otherwise cut to form a tube from the rod.

In accordance with another and/or alternative non-limiting aspect of the present disclosure, all or a portion of the medical device or other type of device that is formed of the metal alloy can be at least partially or fully formed from by 3D printing. As can be appreciated, other portions of the medical device or other type of device can be formed by 3D printing.

In accordance with another and/or alternative non-limiting aspect of the present disclosure, the metal alloy is optionally subjected to a swaging process; however, this is not required. In one non-limiting embodiment, swaging is performed on the metal alloy to at least partially or fully achieve final dimensions of one or more portions of the medical device or other type of device. Where there are undercuts of hollow structures in the medical device, which is not required, a separate piece of metal can be placed in the undercut to at least partially fill the gap. The separate piece of metal, when used, can be designed to be later removed from the undercut; however, this is not required. The swaging operation can be performed on the in areas of the metal alloy to be hardened. For a round or curved portion of a medical device or other device, the swaging can be rotary. For non-round portion of the medical device or other device, the swaging of the non-round portion can be performed by non-rotating swaging dies. The swaging temperature for a particular metal alloy can vary. In one non-limiting embodiment, when the swaging temperature is 10-400° C. and all values and ranges therebetween, the swaging can be conducted in air or an oxidizing environment. When the swaging temperature is increase above 400° C. (e.g., 400-1500+° C. and all values and ranges therebetween) or even when the swaging temperature is at or below 400° C., the swaging process can be performed in a controlled neutral or non-reducing environment (e.g., inert environment). The swaging process can be conducted by repeatedly hammering the metal alloy at the location to be hardened at the desired swaging temperature. In one non-limiting embodiment, during the swaging process, ions of boron and/or nitrogen are allowed to impinge upon atoms in the metal alloy. The medical device can optionally be swaged in multiple directions in a single operation or in multiple operations to achieve a hardness in desired location and/or direction of the medical device. The swaging temperature for a particular metal alloy (e.g., MoRe alloy, ReW alloy, HfW, etc.) can vary. For a refractory alloy (e.g., MoRe alloy, ReW alloy, Hf alloy, Mo alloy, Re alloy, W alloy, etc.) or a metal alloy that includes at least 15 atw. % rhenium and/or hafnium, the swaging temperature can be from room temperature (RT) (e.g., 10-27° C. and all values and ranges therebetween) to about 400° C. (e.g., 10-400° C. and all values and ranges therebetween) if the swaging is conducted in air or an oxidizing environment. The swaging temperature can be increased to up to about 1500° C. (e.g., 10-1500° C. and all values and ranges therebetween) if the swaging process is performed in a controlled neutral or non-reducing environment (e.g., inert environment). The swaging process can be conducted by repeatedly hammering the medical device at the location to be hardened at the desired swaging temperature. In one non-limiting embodiment, when the metal alloy includes rhenium and/or hafnium, during the swaging process ions of boron and/or nitrogen are allowed to impinge upon rhenium and/or hafnium atoms in the MoRe alloy or HfMo alloy or ReHfMo alloy so as to form ReB₂, ReN₂, ReN₃, HfB₂, HfN₂ and/or HrN₃; however, this is not required. It has been found that ReB₂, ReN₂, ReN₃, HfB₂, HfN₂ and/or HrN₃ are ultra-hard compounds. As can be appreciated, other refractory alloys that include Re and/or Hf and that are subjected to a swaging process can also form compounds that include boron and/or nitrogen. In one non-limiting process, the metal for the medical device can be machined and shape into the medical device when the metal is in a less hardened state. As such, the raw starting material can be first annealed to soften and then machined into the metal into a desired shape. After the metal alloy is shaped, the metal alloy can be re-hardened. The hardening of the metal material of the medical device can improve the wear resistance and/or shape retention of the medical device. The metal material of the medical generally cannot be re-hardened by annealing, thus a special rehardening processes is required. Such rehardening can be achieved by the swaging process of the present disclosure.

In accordance with another and/or alternative non-limiting aspect of the present disclosure, the metal alloy can optionally be nitrided; however, this is not required. The nitride layer on the metal alloy can function as a lubricating surface during the optional drawing of the metal alloy, and/or to form a metal alloy that has a smooth and/or lower friction outer surface. After the metal alloy is nitrided, the nitride metal alloy can optionally be cleaned; however, this is not required. The thickness of the nitrided surface layer is generally less than about 1 mm. In one non-limiting embodiment, the thickness of the nitrided surface layer is at least about 50 nanometer and less than about 1 mm (and all values and ranges therebetween). Generally, the weight percent of nitrogen in the nitrided surface layer is 0.0001-5 wt. % nitrogen (and all values and ranges therebetween). In one non-limiting embodiment, the weight percent of nitrogen in the nitrided surface layer is generally less than one of the primary components of the metal alloy, and typically less than each of the two primary components of the metal alloy. For a metal alloy that includes at least 15 atw. % hafnium and/or rhenium, the nitride surface layer typically includes 0.001-5 wt. % nitrogen (and all values and ranges therebetween), and the primary constituents of the metal alloy that includes at least 15 atw. % hafnium and/or rhenium (e.g., metals that constitute at least 5 wt. % of the metal alloy that includes at least 15 atw. % hafnium and/or rhenium) are present in the nitride surface layer in a greater weight percent than the nitrogen content in the metal alloy. The nitriding process for the metal alloy can be used to a) form a lubricating surface, b) increase surface hardness and/or wear resistance of the metal alloy, and/or c) inhibit or prevent discoloration of the metal alloy (e.g., discoloration by oxidation, etc.).

In accordance with another and/or alternative non-limiting aspect of the present disclosure, the metal alloy, just prior to or after being partially or fully formed into the desired medical device or other type of device, can optionally be cleaned, polished, sterilized, nitrided, etc., for final processing of the metal alloy.

In accordance with another and/or alternative non-limiting aspect of the present disclosure, the use of the metal alloy that includes at least 15 atw. % hafnium and/or rhenium, or the refractory metal alloy to partially or fully form the medical device or other type of device can be used to increase the strength, hardness, and/or durability of the medical device or other type of device compared with the same or similar medical device or other type of device that is formed of stainless steel, chromium-cobalt alloys, or titanium alloys; thus, a lesser quantity of metal alloy can be used in the medical device or type of device to achieve similar strengths compared to medical devices or other type of device that is formed of different metals. As such, the resulting medical device or other type of device can be made smaller and/or less bulky by use of the metal alloy without sacrificing the strength and durability of the medical device or other type of device.

In another and/or alternative non-limiting aspect of the present disclosure, the metal alloy includes less than about 5 wt. % (e.g., 0-4.999999 wt. % and all values and ranges therebetween) other metals and/or impurities. A high purity level of the metal alloy results in the formation of a more homogeneous alloy, which in turn results in a more uniform density throughout the metal alloy, and also results in the desired yield and ultimate tensile strengths of the metal alloy. In one non-limiting composition, the metal alloy includes less than about 1 weight percent other metals and/or impurities. In another and/or alternative non-limiting composition, the metal alloy includes less than about 0.5 weight percent other metals and/or impurities. In still another and/or alternative non-limiting composition, the metal alloy includes less than about 0.4 weight percent other metals and/or impurities. In yet another and/or alternative non-limiting composition, the metal alloy includes less than about 0.2 weight percent other metals and/or impurities. In still yet another and/or alternative non-limiting composition, the metal alloy includes less than about 0.1 weight percent other metals and/or impurities. In a another and/or alternative non-limiting composition, the metal alloy includes less than about 0.05 weight percent other metals and/or impurities. In still a another and/or alternative non-limiting composition, the metal alloy includes less than about 0.02 weight percent other metals and/or impurities. In yet a another and/or alternative non-limiting composition, the metal alloy includes less than about 0.01 weight percent other metals and/or impurities. As can be appreciated, other weight percentages of the amount of other metals and/or impurities in the metal alloy can exist.

In another non-limiting aspect of the present disclosure, carbon nanotubes (CNT) can optionally be incorporated into a metal alloy that is used to at least partially form the medical device. The metal alloy can optionally include 0.05 wt. % CNT (e.g., 0.05 wt. % to 10 wt. % and all values and ranges therebetween).

In another and/or alternative non-limiting aspect of the present disclosure, the metal alloy (when used) can be used to form a coating on a portion or all of a medical device. For example, the metal alloy can be used as a coating on a polymer or base metal that is used to form all or a portion of a medical device (e.g., medical device for articulation points of artificial joints, etc.). Such coating can provide the benefit of improved wear, improved scratch resistance, improved hardness, and/or eliminate leaching of harmful metallic ions (i.e., Co, Cr, etc.) from the medical device (e.g., medical devices having articulating surfaces when they undergo fretting (i.e., scratching during relative motion)). As can be appreciated, the metal alloy can have other or additional advantages. As can also be appreciated, the metal alloy can be coated on other or additional types of medical devices. The coating process on a base metal (e.g., Ti or Ti alloy, Co or Co alloy, Cr alloy, CoCr alloy, stainless-steel, Fe and Fe alloys, Ni alloy, W alloy, Mo or Mo alloy, Hf or Hf alloy, Re or Re alloy, etc.) or polymer can be by plasma coating, chemical vapor deposition, and/or 3D printing of the coating on the base metal or polymer. The coating thickness of the metal alloy is non-limiting (e.g., 0.000001-0.5 inches and all values and ranges therebetween). In one non-limiting example, there is provided a medical device in the form of a clad rod wherein in the core of the rod is formed of a metal or ceramic or composite material or polymer, and the outer layer of the rod is formed of the metal alloy that was coated by a plasma coating process, a chemical vapor deposition process, or 3D printing process. The core and the outer coated layer of the rod can each form 50-99% (and all values and ranges therebetween) of the overall cross section of the rod. As can also be appreciated, the metal alloy can form the outer layer of other or additional types of medical devices or devices other than medical devices (e.g., automotive parts, springs, aerospace parts, industrial machinery and parts, tools (e.g., medical tools, industrial tools, household tools, etc.), etc.). The coating can be used to create a hard surface on the medical or devices or than medical devices at specific locations as well as all over the surface. The base hardness of metal alloy can be as low as 300 Vickers and/or as high as 500 Vickers (e.g., 300-500 Vickers and all values and ranges therebetween). However, at high hardness the properties may not be desirable. In instances where the properties of fully annealed material is desired, but only the surface requires to be hardened as in this disclosure, the present disclosure includes a method that can provide benefits of both a softer metal alloy with a harder outer surface or shell. A non-limiting example is an orthopedic screw where a softer iron alloy is desired for high ductility as well as ease of machinability. Simultaneously, a hard shell is desired of the finished screw. While the inner hardness can range from 250 Vickers to 550 Vickers (and all values and ranges therebetween), the outer hardness can vary from 350 Vickers to 1000 Vickers (and all values and ranges therebetween) when using metal alloy.

In another and/or alternative non-limiting aspect of the present disclosure, the metal alloy can be used to form a core of a portion or all of a medical device. For example, a medical device can be in the form of a rod. The core of the rod can be formed of the metal alloy and then the outside of the core can then be coated with one or more other materials (e.g., another type of metal, polymer coating, ceramic coating, composite material coating, etc.). Such a rod can be used, for example, for orthopedic applications such as, but not limited to, spinal rods and/or pedicle screw systems. Non-limiting benefits to using the metal alloy in the core of a medical device can reducing the size of the medical device, increasing the strength of the medical device, and/or maintaining or reducing the cost of the medical device. As can be appreciated, the metal alloy can have other or additional advantages. As can also be appreciated, the metal alloy can form the core of other or additional types of medical devices. The core size and/or thickness of the metal alloy are non-limiting. In one non-limiting example, there is provided a medical device in the form of a clad rod wherein in the core of the rod is formed of a metal alloy, and the other layer of the clad rod is formed of a metal or metal alloy. The core and the other layer of the rod can each form 50-99% (and all values and ranges therebetween) of the overall cross section of the rod. As can also be appreciated, the metal alloy can form the core of other or additional types of medical devices.

In another and/or alternative non-limiting aspect of the present disclosure, the metal alloy has several physical properties that positively affect the medical device when the medical device is at least partially formed of the metal alloy of the present disclosure. In one non-limiting embodiment of the disclosure, the average Vickers hardness of metal alloy of the present disclosure used to form the medical device is generally at least about 234 DHP (Vickers) (i.e., Rockwell A hardness of at least about 60 at 77° F., Rockwell C hardness of at least about 19 at 77° F.) (e.g., 234 DPH to 700 DPH and all values and ranges therebetween; Rockwell C hardness of 19-60 at 77° F. and all values and ranges therebetween); however, this is not required. In one non-limiting aspect of this embodiment, the average hardness of the metal alloy of the present disclosure used to form the medical device is generally at least about 248 DHP (i.e., Rockwell A hardness of at least about 62 at 77° F., Rockwell C hardness of at least about 22 at 77° F.). In another and/or additional non-limiting aspect of this embodiment, the average hardness of the metal alloy of the present disclosure used to form the medical device is generally about 248-513 DHP (i.e., Rockwell A hardness of about 62-76 at 77° F., Rockwell C hardness of about 22-50 at 77° F.). In still another and/or additional non-limiting aspect of this embodiment, the average hardness of the metal alloy of the present disclosure used to form the medical device is generally about 272-458 DHP (i.e., Rockwell A hardness of about 64-74 at 77° F., Rockwell C hardness of about 26-46 at 77° F.). The metal alloy of the present disclosure generally has an average hardness that is greater than stainless-steel. In another and/or alternative non-limiting embodiment of the disclosure, the average ultimate tensile strength of the metal alloy of the present disclosure is generally at least about 60 UTS (ksi); however, this is not required. In one non-limiting aspect of this embodiment, the average ultimate tensile strength of the metal alloy of the present disclosure is generally at least about 70 UTS (ksi) (e.g., 70 UTS to 850 UTS and all values and ranges therebetween), and typically about 80-550 UTS (ksi). The average ultimate tensile strength of the metal alloy of the present disclosure may vary somewhat when the metal alloy is in the form of a tube or a solid wire. When the metal alloy of the present disclosure is in the form of a tube, the average ultimate tensile strength of the metal alloy of the present disclosure is generally about 80-550 UTS (ksi) (and all values and ranges therebetween), typically at least about 110 UTS (ksi), and more typically 110-150 UTS (ksi). When the metal alloy of the present disclosure is in the form of a solid wire, the average ultimate tensile strength of the metal alloy of the present disclosure wire is generally about 120-650 UTS (ksi) (and all values and ranges therebetween). In still another and/or alternative non-limiting embodiment of the disclosure, the average yield strength of the metal alloy of the present disclosure is at least about 70 ksi (e.g., 70-150 ksi and all values and ranges therebetween); however, this is not required. In one non-limiting aspect of this embodiment, the average yield strength of the metal alloy of the present disclosure used to form the medical device is at least about 80 ksi, and typically about 100-150 (ksi). In yet another and/or alternative non-limiting embodiment of the disclosure, the average grain size of the metal alloy of the present disclosure used to form the medical device is no greater than about 4 ASTM (e.g., 4 ASTM to 20 ASTM using ASTM E112 and all values and ranges therebetween, e.g., 0.35 micron to 90 micron, and all values and ranges therebetween); however, this is not required. The grain size as small as about 14-15 ASTM can be achieved; however, the grain size is typically larger than 15 ASTM. The small grain size of the metal alloy of the present disclosure enables the medical device to have the desired elongation and ductility properties that are useful in enabling the medical device to be formed, crimped and/or expanded. In one non-limiting aspect of this embodiment, the average grain size of the metal alloy of the present disclosure used to form the medical device is about 5.2-10 ASTM, typically about 5.5-9 ASTM, more typically about 6-9 ASTM, still more typically about 6-9 ASTM, even more typically about 6.6-9 ASTM, and still even more typically about 7-8.5 ASTM; however, this is not required.

In another and/or alternative non-limiting embodiment of the disclosure, the average tensile elongation of the metal alloy of the present disclosure used to form the medical device is at least about 25% (e.g., 25%-50% average tensile elongation and all values and ranges therebetween). An average tensile elongation of at least 25% for the metal alloy is important to enable the medical device to be properly expanded when positioned in the treatment area of a body passageway. A medical device that does not have an average tensile elongation of at least about 25% can form micro-cracks and/or break during the forming, crimping and/or expansion of the medical device. In one non-limiting aspect of this embodiment, the average tensile elongation of the metal alloy of the present disclosure used to form the medical device is about 25-35%. The unique combination of the metals in the metal alloy of the present disclosure in combination with achieving the desired purity and composition of the alloy and the desired grain size of the metal alloy results in 1) a medical device having the desired high ductility at about room temperature, 2) a medical device having the desired amount of tensile elongation, 3) a homogeneous or solid solution of a metal alloy having high radiopacity, 4) a reduction or prevention of micro-crack formation and/or breaking of the metal alloy of the present disclosure tube when the tube is sized and/or cut to form the medical device, 5) a reduction or prevention of micro-crack formation and/or breaking of the medical device when the medical device is crimped onto a balloon and/or other type of medical device for insertion into a body passageway, 6) a reduction or prevention of micro-crack formation and/or breaking of the medical device when the medical device is bent and/or expanded in a body passageway, 7) a medical device having the desired ultimate tensile strength and yield strength, 8) a medical device that can have very thin wall thicknesses and still have the desired radial forces needed to retain the body passageway on an open state when the medical device has been expanded, and/or 9) a medical device that exhibits less recoil when the medical device is crimped onto a delivery system and/or expanded in a body passageway.

In another and/or alternative non-limiting aspect of the present disclosure, the use of the metal alloy to partially or fully form the medical device can be used to increase the strength and/or hardness and/or durability of the medical device as compared with stainless-steel or chromium-cobalt alloys; thus, less quantity of metal alloy can be used in the medical device to achieve similar strengths as compared to medical devices formed of different metals. As such, the resulting medical device can be made smaller and less bulky by use of the metal alloy without sacrificing the strength and durability of the medical device. Such a medical device can have a smaller profile, thus can be inserted in smaller areas, openings and/or passageways. The metal alloy can also increase the radial strength of the medical device. For instance, the thickness of the walls of the medical device and/or the wires used to form the medical device can be made thinner and achieve a similar or improved radial strength as compared with thicker walled medical devices formed of stainless-steel or cobalt and chromium alloy. The metal alloy also can improve stress-strain properties, bendability and flexibility of the medical device, thus increasing the life of the medical device. For instance, the medical device can be used in regions that subject the medical device to bending. Due to the improved physical properties of the medical device from the metal alloy, the medical device has improved resistance to fracturing in such frequent bending environments. In addition or alternatively, the improved bendability and flexibility of the medical device due to the use of the metal alloy can enable the medical device to be more easily inserted into various regions of a body. The metal alloy can also reduce the degree of recoil during the crimping and/or expansion of the medical device. For example, the medical device better maintains its crimped form and/or better maintains its expanded form after expansion due to the use of the metal alloy. As such, when the medical device is to be mounted onto a delivery device when the medical device is crimped, the medical device better maintains its smaller profile during the insertion of the medical device into various regions of a body. Also, the medical device better maintains its expanded profile after expansion so as to facilitate in the success of the medical device in the treatment area. In addition to the improved physical properties of the medical device by use of the metal alloy, the metal alloy has improved radiopaque properties as compared to standard materials such as stainless-steel or cobalt-chromium alloy, thus reducing or eliminating the need for using marker materials on the medical device. For instance, the metal alloy is believed to be at least about 10-20% more radiopaque than stainless-steel or cobalt-chromium alloy. Specifically, the metal alloy (e.g., novel refractory alloys) is believed to be at least about 33% more radiopaque than cobalt-chromium alloy and is believed to be at least about 40% more radiopaque than stainless-steel.

In accordance with another and/or alternative non-limiting aspect of the present disclosure, the metal alloy or medical device can include, contain and/or be coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. The type and/or amount of agent included in medical device and/or coated on medical device can vary. When two or more agents are included in and/or coated on medical device, the amount of two or more agents can be the same or different. The type and/or amount of agent included on, in and/or in conjunction with medical device are generally selected to address one or more clinical events. As defined herein, an agent is not an enhancement coating as defined herein.

In accordance with another and/or alternative non-limiting aspect of the present disclosure, the amount of agent included on, in and/or used in conjunction with metal alloy or medical device, when the agent is used, is about 0.01-100 ug per mm² (and all values and ranges wherein between) and/or at least about 0.00001 wt. % of device; however, other amounts can be used. The amount of two of more agents on, in and/or used in conjunction with medical device can be the same or different. The one or more agents can be coated on and/or impregnated in medical device by a variety of mechanisms such as, but not limited to, spraying (e.g., atomizing spray techniques, etc.), powder deposition, dip coating, flow coating, dip-spin coating, roll coating (direct and reverse), sonication, brushing, plasma deposition, depositing by vapor deposition, MEMS technology, and rotating mold deposition. The amount of two of more agents on, in and/or used in conjunction with medical device, when two one more agents are used, can be the same or different.

In accordance with another and/or alternative non-limiting aspect of the present disclosure, the one or more agents on and/or in the metal alloy or medical device, when used on the medical device, can be released in a controlled manner so the area in question to be treated is provided with the desired dosage of agent over a sustained period of time.

In accordance with another and/or alternative non-limiting aspect of the present disclosure, the one or more polymers used to at least partially control the release of one or more agents from the metal alloy or medical device can be porous or non-porous. The one or more agents can be inserted into and/or applied to one or more surface structures and/or micro-structures on the metal alloy or medical device, and/or be used to at least partially form one or more surface structures and/or micro-structures on the metal alloy or medical device.

In accordance with another and/or alternative non-limiting aspect of the present disclosure, the thickness of each polymer layer and/or layer of agent is generally at least about 0.01 μm and is generally less than about 150 μm (e.g., 0.01-149.9999 μm and all values and ranges therebetween). In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In accordance with another and/or alternative non-limiting aspect of the present disclosure, a variety of polymers can be coated on the metal alloy or medical device and/or be used to form at least a portion of the medical device. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing and/or the like; however, other or additional coating techniques can be used. The one or more polymers that can be coated on the medical device and/or used to at least partially form the medical device can be polymers that are considered to be biodegradable, bioresorbable, or bioerodable; polymers that are considered to be biostable; and/or polymers that can be made to be biodegradable and/or bioresorbable with modification.

In accordance with another and/or alternative non-limiting aspect of the present disclosure, the medical device can optionally include a marker material that facilitates enabling the medical device to be properly positioned in a body passageway (e.g., blood vessel, heart valve, etc.). The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque).

In accordance with another and/or alternative non-limiting aspect of the present disclosure, the medical device or one or more regions of the medical device can optionally be constructed by use of one or more microelectromechanical manufacturing (MEMS) techniques (e.g., micro-machining, laser micro-machining, laser micro-machining, micro-molding, 3D printing, etc.); however, other or additional manufacturing techniques can be used.

In accordance with another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology (e.g., 3D printing, etc.).

In accordance with another and/or alternative non-limiting aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie-like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm.

In still yet another and/or alternative non-limiting aspect of the present disclosure, the medical device can include and/or be used with a physical hindrance. The physical hindrance can include, but is not limited to, an adhesive, sheath, magnet, tape, wire, string, etc. The physical hindrance can be used to 1) physically retain one or more regions of the medical device in a particular form or profile, 2) physically retain the medical device on a particular deployment device, 3) protect one or more surface structures and/or micro-structures on the medical device, and/or 4) form a barrier between one or more surface regions, surface structures and/or micro-structures on the medical device and the fluids in a body passageway. The physical hindrance is optionally a biodegradable material; however, a biostable material can be used.

In still another and/or alternative aspect of the disclosure, the medical device can be an expandable device that can be expanded by use of some other device (e.g., balloon, etc.) and/or is self-expanding.

In accordance with another and/or alternative non-limiting aspect of the present disclosure, the medical device can optionally be fabricated from a material having no or substantially no shape-memory characteristics.

In accordance with another and/or alternative non-limiting aspect of the present disclosure, there is optionally provided a near net process for a frame, other metal component of the medical device, or a portion or all of another device. In one non-limiting embodiment of the disclosure, there is provided a method of powder pressing materials and optionally increasing the strength post-sintering by imparting additional cold work. In one non-limiting embodiment, the metal alloy powder is pressed into a green part and then sintered. Thereafter, the sintered part is optionally again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered, and re-pressed) meets the dimensional requirements of the final formed part. There is also provided an optional process of increasing the mechanical strength of a pressed metal part by repressing the post-sintered part to add additional cold work into the material, thereby increasing its mechanical strength. There is also provided an optional process of powder pressing to a near net or final part using metal powder. A prepress pressure of 1-300 Tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 Tsi (and all values and ranges therebetween) at a temperature of at least 20° C. (e.g., 20-100° C. and all values and ranges therebetween; 20-40° C., etc.). There is also provided a process of increasing the mechanical strength of a pressed metal part by repressing the post-sintered part to add additional cold work into the material, thereby increasing its mechanical strength. There is also provided a process of powder pressing to a near net or final part using metal powder. The ductility of the metal alloy measured as % reduction in area can increase and yield and ultimate tensile strength can increase.

In accordance with another and/or alternative non-limiting aspect of the present disclosure, there is optionally provided a press of near net or finished part composite (e.g., green part, etc.). The process of pressing metal alloy powder into near net of finished parts is well established; however, pressing a composite structure formed of metal powder and polymer for purposes of making complex part geometries and foam-like structures is new. Similarly, using a pressing process to impart particular biologic substances into the metal matrix is also new. In one non-limiting embodiment, there is provided a process of creating a metal part with pre-defined voids to create a trabecular or foam structure composed of mixing a metal and polymer powder, and then pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation (on the metal substrate) and the polymer residual has some desired biological affect (e.g., masking the metal from the body by encapsulation, promotion of cellular attachment and growth). The polymer and metal powders can be of varying sizes to create a multiplied of voids—some large, creating a pathway for cellular growth, and some small, creating a ruff surface to promote cellular attachment. As such, the use of a polymer in combination with metal powder and subsequent pressing and sintering can be used to form novel and customized shapes for medical device or the near net form of the medical device. Generally, the polymer constitutes about 0.1-70 vol. % (and all values and ranges therebetween) of the consolidated and pressed material prior to the sintering step, typically the polymer constitutes about 1-60 vol. % of the consolidated and pressed material prior to the sintering step, more typically the polymer constitutes about 2-50 vol. % of the consolidated and pressed material prior to the sintering step, and even more typically the polymer constitutes about 2-45 vol. % of the consolidated and pressed material prior to the sintering step. As such, if the polymer constitutes about 5 vol. % of the consolidated and pressed material prior to the sintering step, if after the sintering step at least 95% (e.g., 95-100% and all values and ranges therebetween) of the polymer is degraded and removed from the part or medical device, then the part could include up to about 5 vol. % cavities and/or passageways in the medical device. After the sintering process, at least 95 vol. % (95%-100% and all values and ranges therebetween) of the polymer is thermally degraded and/or removed from the sintered material.

In accordance with another and/or alternative non-limiting aspect of the present disclosure, the metal alloy that is used to at least partially form the medical device or other type of device is initially formed into a near net part, blank, a rod, a tube, etc., and then finished into final form by one or more finishing processes (e.g., centerless grinding, turning, electropolishing, drawing process, grinding, laser cutting, shaving, polishing, EDM cutting, micro-machining, laser micro-machining, micro-molding, machining, drilling (e.g., gun drilling, etc.), 3D printing, cold wording, swaging, cleaning, buffing, smoothing, nitriding, annealing, plug drawing, etching (chemical etching, plasma etching, etc.), chemical modifications, chemical reactions, photo-etching, chemical coatings, etc.).

In accordance with another and/or alternative non-limiting aspect of the present disclosure, the metal alloy that partially or fully forms the near net part, blank, rod, tube, etc., can be formed by various techniques such as, but not limited to, 1) melting the metal alloy (e.g., vacuum arc melting, etc.) and then extruding and/or casting the metal alloy into a near net part, blank, rod, tube, etc., 2) melting the metal alloy, forming a metal strip and then rolling and welding the strip into a near net part, blank, rod, tube, etc., 3) consolidating (pressing, pressing and sintering, etc.) the metal powder of the metal alloy into a near net part, blank, rod, tube, etc., and/or 4) 3D print the metal alloy into a near net part, blank, rod, tube, etc. When the metal alloy is formed into a blank, the shape and size of the blank is non-limiting. When the metal alloy is formed into a rod or tube, the rod or tube generally has a length of about 48 inches or less (e.g., 0.1-48 inches and all values and ranges therebetween); however, longer lengths can be formed. In one non-limiting arrangement, the length of the rod or tube is about 8-20 inches. The average outer diameter of the rod or tube is generally less than about 2 inches (i.e., less than about 3.14 sq. in. cross-sectional area), more typically less than about 1 inch outer diameter, and even more typically no more than about 0.5 inch outer diameter; however, larger rod or tube diameter sizes can be formed. In one non-limiting configuration for a tube, the tube has an inner diameter of about 0.31 inch plus or minus about 0.002 inch and an outer diameter of about 0.5 inch plus or minus about 0.002 inch. The wall thickness of the tube is about 0.095 inch plus or minus about 0.002 inch. As can be appreciated, this is just one example of many different sized tubes that can be formed. In one non-limiting process, the near net medical device, blank, rod, tube, etc. can be formed from one or more ingots of metal or metal alloy.

In one non-limiting process, an arc melting process (e.g., vacuum arc melting process, etc.) can be used to form the near net medical device, blank, rod, tube, etc. In another non-limiting process, metal powder can be placed in a crucible (e.g., silica crucible, etc.) and heated under a controlled atmosphere (e.g., vacuum environment, carbon monoxide environment, hydrogen and argon environment, helium, argon, etc.) by an induction melting furnace to form the near net medical device, blank, rod, tube, etc. It can be appreciated that other or additional processes can be used to form the metal alloy. When a tube of metal alloy is to be formed, a close-fitting rod can be used during the extrusion process to form the tube; however, this is not required. In another and/or additional non-limiting process, the tube of the metal alloy can be formed from a strip or sheet of metal alloy. The strip or sheet of metal alloy can be formed into a tube by rolling the edges of the sheet or strip and then welding together the edges of the sheet or strip. The welding of the edges of the sheet or strip can be accomplished in several ways such as, but not limited to, a) holding the edges together and then e-beam welding the edges together in a vacuum, b) positioning a thin strip of metal alloy above and/or below the edges of the rolled strip or sheet to be welded, then welding the one or more strips along the rolled strip or sheet edges, and then grinding off the outer strip, or c) laser welding the edges of the rolled sheet or strip in a vacuum, oxygen reducing atmosphere, or inert atmosphere. In still another and/or additional non-limiting process, the near net medical device, blank, rod, tube, etc. of the metal alloy is formed by consolidating metal powder. In this process, fine particles of metal (e.g., Re, Hf, W, Mo, Ti, Cu, Ni, Cr, etc.) along with any additives are mixed to form a homogenous blend of particles. Typically, the average particle size of the metal powders is less than about 200 mesh (e.g., less than 74 microns). A larger average particle size can interfere with the proper mixing of the metal powders and/or adversely affect one or more physical properties of the near net medical device, blank, rod, tube, etc. formed from the metal powders. In one non-limiting embodiment, the average particle size of the metal powders is less than about 230 mesh (e.g., less than 63 microns). In another and/or alternative non-limiting embodiment, the average particle size of the metal powders is about 2-63 microns, and more particularly about 5-40 microns. As can be appreciated, smaller average particle sizes can be used. The purity of the metal powders should be selected so that the metal powders contain very low levels of carbon, oxygen and nitrogen. Typically, the carbon content of the metal powder used to form the metal alloy is less than about 100 ppm, the oxygen content is less than about 50 ppm, and the nitrogen content is less than about 20 ppm. Typically, metal powder used to form the metal alloy has a purity grade of at least 99.9 and more typically at least about 99.95. The blend of metal powder is then pressed together to form a solid solution of the metal alloy into a near net medical device, blank, rod, tube, etc. Typically the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen reducing atmosphere (e.g., hydrogen, argon and hydrogen mixture, etc.) and/or under a vacuum; however, this is not required. The average density of the near net medical device, blank, rod, tube, etc. that is achieved by pressing together the metal powders is about 80-95% (and all values and ranges therebetween) of the final average density of the near net medical device, blank, rod, tube, etc. or about 70-96% (and all values and ranges therebetween) the minimum theoretical density of the metal alloy. Pressing pressures of at least about 300 MPa are generally used. Generally, the pressing pressure is about 400-700 MPa; however, other pressures can be used. After the metal powders are pressed together, the pressed metal powders are sintered at a temperature of at least 1600° C. (e.g., 1600-3500° C. and all values and ranges therebetween) to partially or fully fuse the metal powders together to form the near net medical device, blank, rod, tube, etc. The sintering of the consolidated metal powder can be performed in an oxygen reducing atmosphere (e.g., helium, argon, hydrogen, argon and hydrogen mixture, etc.) and/or under a vacuum; however, this is not required. At the high sintering temperatures, a high hydrogen atmosphere will reduce both the amount of carbon and oxygen in the formed near net medical device, blank, rod, tube, etc. The sintered metal powder generally has an as-sintered average density of about 90-99% the minimum theoretical density of the metal alloy. Typically, the sintered metal alloy has a final average density of at least about 5 gm/cc, and typically at least about 8.3 gm/cc, and can be up to or greater than about 16 gm/cc; however, this is not required. The density of the formed near net medical device, blank, rod, tube, etc. will generally depend on the type of metal alloy used.

In accordance with another and/or alternative non-limiting aspect of the present disclosure, when a solid rod of the metal alloy is formed, the rod can optionally be formed into a tube prior to reducing the outer cross-sectional area or diameter of the rod. The rod can be formed into a tube by a variety of processes such as, but not limited to, cutting or drilling (e.g., gun drilling, etc.) or by cutting (e.g., EDM, EDM sinker, wire EDM, etc.) or by 3D printing. The cavity or passageway formed in the rod typically is formed fully through the rod; however, this is not required.

In yet a another and/or alternative non-limiting aspect of the present disclosure, the near net medical device, blank, rod, tube, etc., can optionally be cleaned and/or polished after the near net medical device or other type of device, blank, rod, tube, etc., has been form; however, this is not required. Typically, the near net medical device or other type of device, blank, rod, tube, etc., is cleaned and/or polished prior to being further processed; however, this is not required.

In still yet a another and/or alternative non-limiting aspect of the present disclosure, the near net medical device, blank, rod, tube, etc. can be resized to the desired dimension of the medical device. In one non-limiting embodiment, the cross-sectional area or diameter of the near net medical device, blank, rod, tube, etc. is reduced to a final near net medical device, blank, rod, tube, etc. dimension in a single step or by a series of steps. The reduction of the outer cross-sectional area or diameter of the near net medical device, blank, rod, tube, etc. may be obtained by centerless grinding, turning, electropolishing, drawing process, grinding, laser cutting, shaving, polishing, EDM cutting, etc. The outer cross-sectional area or diameter size of the near net medical device, blank, rod, tube, etc. can be reduced by the use of one or more drawing processes; however, this is not required. During the drawing process, care should be taken to not form micro-cracks in the near net medical device, blank, rod, tube, etc. during the reduction of the near net medical device, blank, rod, tube, etc. outer cross-sectional area or diameter. Generally, the near net medical device, blank, rod, tube, etc. should not be reduced in cross-sectional area by more about 25% each time the near net medical device, blank, rod, tube, etc. is drawn through a reducing mechanism (e.g., a die, etc.). In one non-limiting process step, the near net medical device, blank, rod, tube, etc. is reduced in cross-sectional area by about 0.1-20% each time the near net medical device, blank, rod, tube, etc. is drawn through a reducing mechanism. In another and/or alternative non-limiting process step, the near net medical device, blank, rod, tube, etc. is reduced in cross-sectional area by about 1-15% each time the near net medical device, blank, rod, tube, etc. is drawn through a reducing mechanism. In still another and/or alternative non-limiting process step, the near net medical device, blank, rod, tube, etc. is reduced in cross-sectional area by about 2-15% each time the near net medical device, blank, rod, tube, etc. is drawn through reducing mechanism. In yet another one non-limiting process step, the near net medical device, blank, rod, tube, etc. is reduced in cross-sectional area by about 5-10% each time the near net medical device, blank, rod, tube, etc. is drawn through reducing mechanism. In another and/or alternative non-limiting embodiment of the disclosure, the near net medical device, blank, rod, tube, etc. of metal alloy is drawn through a die to reduce the cross-sectional area of the near net medical device, blank, rod, tube, etc. Generally, before drawing the near net medical device, blank, rod, tube, etc. through a die, one end of the near net medical device, blank, rod, tube, etc. is narrowed down (nosed) so as to allow it to be fed through the die; however, this is not required. The tube drawing process is typically a cold drawing process or a plug drawing process through a die. When a cold drawing or mandrel drawing process is used, a lubricant (e.g., molybdenum paste, grease, etc.) is typically coated on the outer surface of the near net medical device, blank, rod, tube, etc. and the near net medical device, blank, rod, tube, etc. is then drawn though the die. Typically, little or no heat is used during the cold drawing process. After the near net medical device, blank, rod, tube, etc. has been drawn through the die, the outer surface of the near net medical device, blank, rod, tube, etc. is typically cleaned with a solvent to remove the lubricant so as to limit the amount of impurities that are incorporated in the metal alloy; however, this is not required. This cold drawing process can be repeated several times until the desired outer cross-sectional area or diameter, inner cross-sectional area or diameter and/or wall thickness of the near net medical device, blank, rod, tube, etc. is achieved. A plug drawing process can also or alternatively be used to size the near net medical device, blank, rod, tube, etc. The plug drawing process typically does not use a lubricant during the drawing process. The plug drawing process typically includes a heating step to heat the near net medical device, blank, rod, tube, etc. prior and/or during the drawing of the near net medical device, blank, rod, tube, etc. through the die. The elimination of the use of a lubricant can reduce the incidence of impurities being introduced into the metal alloy during the drawing process. During the plug drawing process, the near net medical device, blank, rod, tube, etc. can be protected from oxygen by use of a vacuum environment, a non-oxygen environment (e.g., hydrogen, argon and hydrogen mixture, nitrogen, nitrogen and hydrogen, etc.) or an inert environment. One non-limiting protective environment includes argon, hydrogen or argon and hydrogen; however, other or additional inert gasses can be used. As indicated above, the near net medical device, blank, rod, tube, etc. is typically cleaned after each drawing process to remove impurities and/or other undesired materials from the surface of the near net medical device, blank, rod, tube, etc.; however, this is not required. Typically, the near net medical device, blank, rod, tube, etc. should be shielded from oxygen and nitrogen when the temperature of the near net medical device, blank, rod, tube, etc. is increased to above 500° C., and typically above 450° C., and more typically above 400° C.; however, this is not required. When the near net medical device, blank, rod, tube, etc. is heated to temperatures above about 400-500° C., the near net medical device, blank, rod, tube, etc. tends to begin forming nitrides and/or oxides in the presence of nitrogen and oxygen. In these higher temperature environments, a hydrogen environment, an argon and hydrogen environment, etc. is generally used. When the near net medical device, blank, rod, tube, etc. is drawn at temperatures below 400-500° C., the near net medical device, blank, rod, tube, etc. can be exposed to air with little or no adverse effects; however, an inert or slightly reducing environment is generally more desirable.

In another and/or alternative non-limiting aspect of the present disclosure, a rod of metal alloy is partially or fully reduced to the desired outer diameter prior to forming a tube from the rod. Once the solid metal rod is formed, the outer diameter of the metal rod is reduced by cold working the rod using or more techniques such as, but not limited to, use of a mandrel, use of one or more rollers, use of an extrusion process, use of a die, squeezing processes, cold rolling process, a rotary swaging process, cold forging process, sizing processes, drawing processes, etc. The rod can be processed one or more times using one or more cold working techniques prior to achieving the desired outer diameter of the rod. Generally, the rod is not reduced in cross-sectional area by more about 25% each time the rod is subjected to a reducing mechanism. During the reducing process, the solid rod can be annealed one or more times to facilitate in the reduction of the outer diameter of the rod. The rod may or may not be annealed each time the rod is subjected to a reducing mechanism. Once the solid rod has obtained it desired outer diameter, the solid rod is typically not further annealed so as to maintain the hardness properties and yield strength properties that were obtained by the cold working of the rod. The above discussed parameters for reducing the outer diameter of the solid rod to the desired outer diameter can be used. Once the solid rod has the desired outer diameter, the solid rod is formed into a tube by gun drilling the rod or EDM cutting of the rod. Further processing of the tube can occur to finalize the wall thickness of the tube and uniformity of the wall thickness of the tube. One non-limiting process that can be used is a wire EDM machining process. Once the tube is formed, the tube can be subject to one or more treatment processes such as cleaning, polishing, sterilizing, nitrided, etc. for final processing of the metal tube. The metal tube can be further processed by cutting the metal tube into a portion of or the complete final form (e.g., final medical device—stent, TAVR, screw, etc.). It has been found that metal tubes that are first formed by reducing the outer diameter of the rod and then hollowing out the rod to form a tube have a final yield strength of at least 30% greater than tubes that are first formed and then the outer diameter of the tube is reduced. For example, metal alloy tubes that have yield strengths of 130 Ksi when formed from a tube that has been reduced in outer diameter (and not annealed after achieving its final outer diameter) can have yield strengths of at least 200 Ksi when formed by first reducing a solid rod by a cold working process and then hollowing out the solid rod to form a tube after the rod has been reduced to the desired outer diameter.

In accordance with another and/or alternative aspect of the present disclosure, when metal powder is used to 3D print rod, tube, etc., the average particle size of the metal powder is optionally 2-62 microns, and more particularly about 5-49.9 microns, the average density of the metal powders is greater than 5 g/cm³, and the metal powder is generally spherical-shaped, and the Hall flow (s/50 g) is less than 30 seconds (e.g., 2-29.99 seconds and all values and ranges therebetween).

In still a another and/or alternative non-limiting aspect of the present disclosure, the near net medical device, blank, rod, tube, etc. during the drawing process can be nitrided; however, this is not required. The nitride layer on the near net medical device, blank, rod, tube, etc. can function as a lubricating surface during the drawing process to facilitate in the drawing of the near net medical device, blank, rod, tube, etc. The near net medical device, blank, rod, tube, etc. is generally nitrided in the presence of nitrogen or a nitrogen mixture. In one non-limiting embodiment of the disclosure, the surface of the near net medical device, blank, rod, tube, etc. is nitrided prior to at least one drawing step for the near net medical device, blank, rod, tube, etc. In one non-limiting aspect of this embodiment, the surface of the near net medical device, blank, rod, tube, etc. is nitrided prior to a plurality of drawing steps. In another non-limiting aspect of this disclosure, after the near net medical device, blank, rod, tube, etc. has been annealed, the near net medical device, blank, rod, tube, etc. is nitrided prior to being drawn. In another and/or alternative non-limiting embodiment, the near net medical device, blank, rod, tube, etc. is cleaned to remove nitride compounds on the surface of the near net medical device, blank, rod, tube, etc. prior to annealing the rod to tube. The nitride compounds can be removed by a variety of steps such as, but not limited to, grit blasting, polishing, etc. After the near net medical device, blank, rod, tube, etc. has been annealed, the near net medical device, blank, rod, tube, etc. can be again nitrided prior to one or more drawing steps; however, this is not required. As can be appreciated, the complete outer surface of the near net medical device, blank, rod, tube, etc. can be nitrided or a portion of the outer surface of the near net medical device, blank, rod, tube, etc. can be nitrided. Nitriding only selected portions of the outer surface of the near net medical device, blank, rod, tube, etc. can be used to obtain different surface characteristics of the near net medical device, blank, rod, tube, etc.; however, this is not required.

In still yet a another and/or alternative non-limiting aspect of the present disclosure, the near net medical device, blank, rod, tube, etc. is annealed after one or more drawing processes. The metal alloy blank, rod, tube, etc. can be annealed after each drawing process or after a plurality of drawing processes. The metal alloy blank, rod, tube, etc. is typically annealed prior to about a 60% cross-sectional area size reduction of the metal alloy blank, rod, tube, etc. In other words, the near net medical device, blank, rod, tube, etc. should not be reduced in cross-sectional area by more than 60% before being annealed (e.g., 0.1-60% reduction and all values and ranges therebetween). A too-large reduction in the cross-sectional area of the metal alloy blank, rod, tube, etc. during the drawing process prior to the near net medical device, blank, rod, tube, etc. being annealed can result in micro-cracking of the near net medical device, blank, rod, tube, etc. In one non-limiting processing step, the metal alloy blank, rod, tube, etc. is annealed prior to about a 50% cross-sectional area size reduction of the metal alloy blank, rod, tube, etc. In another and/or alternative non-limiting processing step, the metal alloy blank, rod, tube, etc. is annealed prior to about a 45% cross-sectional area size reduction of the metal alloy blank, rod, tube, etc. In still another and/or alternative non-limiting processing step, the metal alloy blank, rod, tube, etc. is annealed prior to about a 1-45% cross-sectional area size reduction of the metal alloy blank, rod, tube, etc. In yet another and/or alternative non-limiting processing step, the metal alloy blank, rod, tube, etc. is annealed prior to about a 5-30% cross-sectional area size reduction of the metal alloy blank, rod, tube, etc. In still yet another and/or alternative non-limiting processing step, the metal alloy blank, rod, tube, etc. is annealed prior to about a 5-15% cross-sectional area size reduction of the metal alloy blank, rod, tube, etc.

In another and/or alternative non-limiting aspect of the disclosure, when the near net medical device, blank, rod, tube, etc. is annealed, the near net medical device, blank, rod, tube, etc. is typically heated to a temperature of about 500-1700° C. (and all values and ranges therebetween) for a period of about 1-200 minutes (and all values and ranges therebetween); however, other temperatures and/or times can be used. In one non-limiting processing step, the near net medical device, blank, rod, tube, etc. is annealed at a temperature of about 1000-1600° C. for about 2-100 minutes. In another non-limiting processing step, the near net medical device, blank, rod, tube, etc. is annealed at a temperature of about 1100-1500° C. for about 5-30 minutes. The annealing process typically occurs in an inert environment or an oxygen-reducing environment so as to limit the amount of impurities that may embed themselves in the metal alloy during the annealing process. One non-limiting oxygen-reducing environment that can be used during the annealing process is a hydrogen environment; however, it can be appreciated that a vacuum environment can be used or one or more other or additional gasses can be used to create the oxygen-reducing environment. At the annealing temperatures, a hydrogen-containing atmosphere can further reduce the amount of oxygen in the near net medical device, blank, rod, tube, etc. The chamber in which the near net medical device, blank, rod, tube, etc. is annealed should be substantially free of impurities (e.g., carbon, oxygen, and/or nitrogen) so as to limit the amount of impurities that can embed themselves in the near net medical device, blank, rod, tube, etc. during the annealing process.

In another and/or alternative non-limiting aspect of the present disclosure, the parameters for annealing can be changed as the near net medical device, blank, rod, tube, etc. as the cross-sectional area or diameter; and/or wall thickness of the near net medical device, blank, rod, tube, etc. are changed. It has been found that good grain size characteristics of the near net medical device, blank, rod, tube, etc. can be achieved when the annealing parameters are varied as the parameters of the near net medical device, blank, rod, tube, etc. change. For example, as the wall thickness is reduced, the annealing temperature is correspondingly reduced; however, the times for annealing can be increased. As can be appreciated, the annealing temperatures of the near net medical device, blank, rod, tube, etc. can be decreased as the wall thickness decreases, but the annealing times can remain the same or also be reduced as the wall thickness reduces. After each annealing process, the grain size of the metal in the near net medical device, blank, rod, tube, etc. should be no greater than 4 ASTM. Generally, the grain size range is about 4-20 ASTM (and all values and ranges therebetween). It is believed that as the annealing temperature is reduced as the wall thickness reduces, small grain sizes can be obtained. The grain size of the metal in the near net medical device, blank, rod, tube, etc. should be as uniform as possible. In addition, the sigma phase of the metal in the near net medical device, blank, rod, tube, etc. should be as reduced as much as possible. The sigma phase is a spherical, elliptical or tetragonal crystalline shape in the metal alloy.

In another and/or alternative non-limiting aspect of the present disclosure, after the final drawing of the near net medical device, blank, rod, tube, etc., a final annealing of the near net medical device, blank, rod, tube, etc. can be done for final strengthening of the near net medical device, blank, rod, tube, etc.; however, this is not required. This final annealing process, when used, generally occurs at a temperature of about 500-1600° C. (and all values and ranges therebetween) for at least about 1 minutes; however, other temperatures and/or time periods can be used.

In another and/or alternative non-limiting aspect of the present disclosure, the near net medical device or other type of device, blank, rod, tube, etc. general if not reduced in cross-sectional area by more about 25% (e.g., 0.1-25% and all values and ranges therebetween) each time the near net medical device or other type of device, blank, rod, tube, etc. is drawn down in size.

In yet a another and/or alternative non-limiting aspect of the present disclosure, the near net medical device, blank, rod, tube, etc. is cooled after being annealed; however, this is not required. Generally, the near net medical device, blank, rod, tube, etc. is cooled at a fairly quick rate after being annealed so as to inhibit or prevent the formation of a sigma phase in the metal alloy; however, this is not required. Generally, the near net medical device, blank, rod, tube, etc. is cooled at a rate of at least about 50° C. per minute (e.g., 50-500° C. per minute and all values and ranges therebetween) after being annealed, typically at least about 75° C. per minute after being annealed, more typically at least about 100° C. per minute after being annealed, even more typically about 100-400° C. per minute after being annealed, still even more typically about 150-350° C. per minute after being annealed, and yet still more typically about 200-300° C. per minute after being annealed, and still yet even more typically about 250-280° C. per minute after being annealed; however, this is not required.

In accordance with another and/or alternative non-limiting aspect of the present disclosure, the near net medical device or other type of device, blank, rod, tube, etc. can be cleaned prior to and/or after being annealed.

In another and/or alternative non-limiting aspect of the present disclosure, the near net medical device, blank, rod, tube, etc. can be cleaned prior to and/or after being annealed. The cleaning process is designed to remove impurities, lubricants (e.g., nitride compounds, molybdenum paste, grease, etc.) and/or other materials from the surfaces of the near net medical device, blank, rod, tube, etc. Impurities that are on one or more surfaces of the near net medical device, blank, rod, tube, etc. can become permanently embedded into the near net medical device, blank, rod, tube, etc. during the annealing processes. These imbedded impurities can adversely affect the physical properties of the metal alloy as the near net medical device, blank, rod, tube, etc. is formed into a medical device, and/or can adversely affect the operation and/or life of the medical device. In one non-limiting embodiment of the disclosure, the cleaning process includes a delubrication or degreasing process which is typically followed by pickling process; however, this is not required. The delubrication or degreasing process followed by pickling process is typically used when a lubricant has been used on the near net medical device, blank, rod, tube, etc. during a drawing process. Lubricants commonly include carbon compounds, nitride compounds, molybdenum paste, and other types of compounds that can adversely affect the metal alloy if such compounds and/or elements in such compounds become associated and/or embedded with the metal alloy during an annealing process. The delubrication or degreasing process can be accomplished by a variety of techniques such as, but not limited to, 1) using a solvent (e.g., acetone, methyl alcohol, etc.) and wiping the metal alloy with a Kimwipe or other appropriate towel, 2) by at least partially dipping or immersing the metal alloy in a solvent and then ultrasonically cleaning the metal alloy, 3) sand blasting the metal alloy, and/or 4) chemical etching the metal alloy. As can be appreciated, the metal alloy can be delubricated or degreased in other or additional ways. After the near net medical device, blank, rod, tube, etc. has been delubricated or degreased, the near net medical device, blank, rod, tube, etc. can be further cleaned by use of a pickling process; however, this is not required. The pickling process (when used) includes the use of one or more acids to remove impurities from the surface of the near net medical device, blank, rod, tube, etc. Non-limiting examples of acids that can be used as the pickling solution include, but are not limited to, nitric acid, acetic acid, sulfuric acid, hydrochloric acid, and/or hydrofluoric acid. These acids are typically analytical reagent (ACS) grade acids. The acid solution and acid concentration are selected to remove oxides and other impurities on the near net medical device, blank, rod, tube, etc. surface without damaging or over-etching the surface of the near net medical device, blank, rod, tube, etc.

In accordance with another and/or alternative non-limiting aspect of the present disclosure, the near net medical device or other type of device, blank, rod, tube, etc., after a) being formed to the desired green shape, b) after being formed to have the desired outer cross-sectional area or diameter, and/or c) after being formed to have the desired inner cross-sectional area or diameter and/or wall thickness, can then be cut and/or etched to at least partially form the desired configuration of the medical device (e.g., stent, frame for TAV valve, orthopedic device, vascular device, etc.). The near net medical device or other type of device, blank, rod, tube, etc. can be cut or otherwise formed by one or more processes (e.g., centerless grinding, turning, electropolishing, drawing process, grinding, laser cutting, shaving, polishing, EDM cutting, etching, micro-machining, laser micro-machining, micro-molding, machining, etc.). As can be appreciated, a portion or all of the medical device or other type of device can be formed by 3D printing.

In still another and/or alternative non-limiting aspect of the present disclosure, the metal alloy, after being formed to the desired shape, the outer cross-sectional area or diameter, inner cross-sectional area or diameter and/or wall thickness, can be cut and/or etched to at least partially form the desired configuration of the medical device (e.g., stent, TAVR valve frame, pedicle screw, PFO device, spinal implant, vascular implant, rod, guide wire, sheath frame, stent hypotube, etc.). The near net medical device, blank, rod, tube, etc. can be cut or otherwise formed by one or more processes (e.g., centerless grinding, turning, electropolishing, drawing process, grinding, laser cutting, shaving, polishing, EDM cutting, etching, micro-machining, laser micro-machining, micro-molding, machining, etc.). In one non limiting embodiment of the disclosure, the metal alloy near net medical device, blank, rod, tube, etc. is at least partially cut by a laser. The laser is typically desired to have a beam strength which can heat the metal alloy near net medical device, blank, rod, tube, etc. to a temperature up to at least about 2200-2300° C. In one non-limiting aspect of this embodiment, a pulsed Nd:YAG neodymium-doped yttrium aluminum garnet (Nd:Y₃Al₅O₁₂) or CO₂ laser is used to at least partially cut a pattern of a medical device out of the metal alloy blank, rod, tube, etc. In another and/or alternative non-limiting aspect of this embodiment, the cutting of the metal alloy near net medical device, blank, rod, tube, etc. by the laser can occur in a vacuum environment, an oxygen-reducing environment, or an inert environment; however, this is not required. It has been found that laser cutting of the near net medical device, blank, rod, tube, etc. in a non-protected environment can result in impurities being introduced into the cut near net medical device, blank, rod, tube, etc., which introduced impurities can induce micro-cracking of the near net medical device, blank, rod, tube, etc. during the cutting of the near net medical device, blank, rod, tube, etc. One non-limiting oxygen-reducing environment includes a combination of argon and hydrogen; however, a vacuum environment, an inert environment, or other or additional gasses can be used to form the oxygen reducing environment. In still another and/or alternative non-limiting aspect of this embodiment, the metal alloy near net medical device, blank, rod, tube, etc. is stabilized so as to limit or prevent vibration of the near net medical device, blank, rod, tube, etc. during the cutting process. Vibrations in the near net medical device, blank, rod, tube, etc. during the cutting of the near net medical device, blank, rod, tube, etc. can result in the formation of micro-cracks in the near net medical device, blank, rod, tube, etc. as the near net medical device, blank, rod, tube, etc. is cut. The average amplitude of vibration during the cutting of the near net medical device, blank, rod, tube, etc. is generally no more than about 150% (e.g., 0-150% and all values and ranges therebetween) of the wall thickness of the near net medical device, blank, rod, tube, etc.; however, this is not required.

In accordance with another and/or alternative non-limiting aspect of the present disclosure, the metal alloy can be coated with an enhancement coating to improve one or more properties of the metal alloy (e.g., change exterior color of metal alloy, increase hardness of coated surface, increase toughness of coated surface, reduced friction to coated surface, improve impact wear of coated surface, improve resistance to corrosion and oxidation, form a non-stick coated surface, improve biocompatibility of metal alloy having the coated surface, reduce toxicity of metal alloy having the coated surface, etc.). In some vascular applications, the use of the enhancement coating one or more portions of the metal alloy that forms all or a portion of the medical device (e.g., stent, frame of a heart valve, etc.) and/or on a portion of the medical device that does not includes the metal alloy (e.g., leaflet of a heart valve, outer skirt of a heart valve, inner skirt of a heart valve, etc.), the enhancement coating, after the medical device has been implanted into the vascular system, can inhibit or prevent calcium deposits on one or more portions of the medical device (e.g., inhibit or prevent calcium deposits on frame, leaflets, skirt, etc.). Such reduction in calcium deposits on the medical device can a) extend the life of the medical device, and/or b) inhibit or prevent interference with the proper operation of the medical device. The enhancement coating can be applied prior to or after the optional further processing of the medical device or other type of device into its final form. For example, if a rod or tube of metal alloy is to be further cut, etched, nitrided, swaged, heat treated, drawing down, bent, etc. to form all or a portion of the medical device or other type of device, the enhancement coating can be applied to the tube or rod after such cutting, etching, nitriding, swaging, heat treating, drawing down, bending, etc. In one non-limiting embodiment, a portion or all of the medical device or other type of device is formed of a metal alloy, and wherein a portion or all of the outer surface of the metal alloy is coated with an enhancement coating (e.g., chromium nitride (CrN), diamond-like carbon (DLC), titanium nitride (TiN), zirconium nitride (ZrN), zirconium oxide (ZrO2), zirconium-nitrogen-carbon (ZrNC), zirconium OxyCarbide (ZrOC), and wherein the outer surface of the metal alloy optionally includes an adhesion layer, which adhesion layer is optionally a metallic layer that includes titanium or zirconium. In another non-limiting embodiment, the metal alloy is coated with an enhancement coating, and wherein the outer surface of the metal alloy optionally includes an adhesion layer, which adhesion layer is optionally a metallic layer that includes titanium or zirconium.

Another and/or alternative non-limiting aspect of the present disclosure is the provision of a of a medical device or other type of device that is coated with an enhancement layer (e.g., metal oxynitride layer) that facilitates in the formation of a) nitric oxide (NO) production, b) stimulation of endothelial cells, and/or c) a modulation of endothelial cells. Non-limiting enhancement coatings that can be applied to a portion of all of the outer surface of the medical device or other type of device includes chromium nitride (CrN), diamond-like carbon (DLC), titanium nitride (TiN), titanium oxynitride or titanium nitride oxide (TiNOx), zirconium nitride (ZrN), zirconium oxide (ZrO2), zirconium oxynitride (ZrNxOy) [e.g., cubic ZrN:O, cubic ZrO2:N, tetragonal ZrO2:N, and monoclinic ZrO2:N phase coatings], oxyzirconium-nitrogen-carbon (ZrNC), zirconium OxyCarbide (ZrOC), and combinations of such coatings. In one non-limiting embodiment, the one or more enhancement coatings are applied to a portion of all of the outer surface of the metal alloy that includes at least 15 atw. % hafnium can be a vacuum process using an energy source to vaporize material and deposit a thin layer of enhancement coating material. Such vacuum coating process includes a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400° C. (and all values and ranges therebetween) for at least 10 minutes (e.g., 10-400 minutes and all values and ranges therebetween). In another non-limiting embodiment, the coating process is one or more of a PVD, CVD, ALD and PE-CVD, and wherein the coating process occurs at a temperature of 220-300° C. for 60-120 minutes. The materials of the one or more enhancement coatings can be combine with one or more metals in the metal alloy, and/or combined with nitrogen, oxygen, carbon, or other elements that are in the metal alloy and/or present in the atmosphere about the metal alloy to a form an enhancement coating on the outer surface of the metal alloy that can have enhanced properties (e.g., enhancement coating is harder than case-hardened steel, enhancement coating is more scratch-resistant than hardened chrome, enhancement coating having high corrosion resistance, etc.). In another non-limiting embodiment, the one or more enhancement coatings can form various coating colors on the outer surface of the metal alloy (e.g., gold, copper, brass, black, rose gold, chrome, blue, silver, yellow, green, etc.). In another non-limiting embodiment, the thickness of the enhancement coating is greater than 1 nanometer (e.g., 2 nanometers to 100 microns and all values and ranges therebetween), and typically 0.1-25 microns, and more typically 1-10 microns. In another non-limiting embodiment, the hardness of the enhancement coating is at 5 GPa (ASTM C1327-15 or ASTM C1624-05), typically 5-50 GPa (and all values and ranges therebetween), more typically 10-25 GPa, and still more typically 14-24 GPa. In another non-limiting embodiment, the coefficient of friction (COF) of the enhancement coating is 0.04-0.2 (and all values and ranges therebetween), and typically 0.6-0.15. In another non-limiting embodiment, the wear rate of the enhancement coating is 0.5×10⁻⁷ mm³/N-m to 3×10⁻⁷ mm³/N-m (and all values and ranges therebetween), and typically 1.2×10⁻⁷ mm³/N-m to 2×10⁻⁷ mm³/N-m. In another non-limiting embodiment, silicon-based precursors (e.g., trimethysilane, tetramethylsilane, hexachlorodisilane, silane, dichlorosilane, trichlorosilane, silicon tetrachloride, tris(dimethylamino) silane, bis(tert-butylamino)silane, trisilylamine, allyltrimethoxysilane, (3-aminopropyl)triethoxysilane, butyltrichlorosilane, n-sec-butyl(trimethylsilyl)amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohexasilane, hexamethyldisilane, isobutyl(trimethoxy)silane, methyltrichlorosilane, 2,4,6,8,10-pentamethylcyclopentasiloxane, pentamethyldisilane, n-propyltriethoxysilane, silicon tetrabromide, silicon tetrabromide, etc.) can be used to facilitate in the application of the enhancement coating to one or more portions or all of the outer surface of the metal alloy. In one non-limiting embodiment, the enhancement coating optionally includes no more than 0.1 wt. % nickel, no more than 0.1 wt. % chromium, and/or no more than 0.1 wt. % cobalt. In another non-limiting embodiment, the outer surface of the medical device or other type of device optionally includes no more than 0.1 wt. % nickel, no more than 0.1 wt. % chromium, and/or no more than 0.1 wt. % cobalt. The adhesion layer optionally includes no more than 0.1 wt. % nickel, no more than 0.1 wt. % chromium, and/or no more than 0.1 wt. % cobalt. The metal alloy that forms a portion or all of the medical device or other type of device optionally includes no more than 0.1 wt. % nickel, no more than 0.1 wt. % chromium, and/or no more than 0.1 wt. % cobalt.

Another and/or alternative non-limiting object of the present disclosure is the provision of a medical device or other type of device wherein the enhancement coating is partially or fully applied to a metallic adhesion layer. In one non-limiting embodiment, the metallic adhesion layer optionally includes titanium metal or zirconium metal. In another on-limiting embodiment, the metallic adhesion layer optionally has a thickness of 1 to 500 nanometers (and all values and ranges therebetween). The enhancement coating and/or the metallic adhesion layer can be applied by use of a vacuum coating process (e.g., physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process), plating process, etc.

In accordance with another and/or alternative non-limiting aspect of the present disclosure, the metal alloy is coated with an enhancement coating to improve one or more properties of the metal alloy wherein the enhancement coating composition includes a chromium nitride (CrN) coating. A portion or all of the outer surface of the metal alloy can include the chromium nitride (CrN) coating. The enhancement coating can be used to improve hardness, improve toughness, reduced friction, resistant impact wear, improve resistance to corrosion and oxidation, and/or form a reduced stick surface when in contact with many different materials. In accordance with one non-limiting embodiment, the metal alloy is coated with an enhancement coating that generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, all or a portion of the outer surface of the metal alloy is initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of the metal alloy. In another non-limiting embodiment, the enhancement coating composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes a diamond-Like Carbon (DLC) coating. A portion or all of the outer surface of the metal alloy can include the diamond-Like Carbon (DLC) coating. The enhancement coating can be used to improve hardness, improve toughness, reduced friction, resistant impact wear, improve resistance to corrosion and oxidation, improve biocompatibility, and/or form a reduced stick surface when in contact with many different materials. In one non-limiting embodiment, all or a portion of the outer surface of the metal alloy is coated with the enhancement coating composition that generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by using methane and/or acetylene gas; however, other or additional carbon sources can be used. The coating thickness of the carbon is 0.5-15 microns. In another non-limiting embodiment, all or a portion of the outer surface of the metal alloy is coated with the enhancement coating composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, the metal alloy is coated with an enhancement coating to improve one or more properties of the metal alloy wherein the enhancement coating composition generally includes a titanium nitride (TiN) coating. A portion or all of the outer surface of the metal alloy can include the titanium nitride (TiN) coating. The enhancement coating can be used to improve hardness, improve toughness, improve resistance to corrosion and oxidation, reduced friction, and/or form a reduced stick surface when in contact with many different materials. In one non-limiting embodiment, all or a portion of the outer surface of the metal alloy is initially coated with Ti metal, which Ti metal is a component of the enhancement coating. The Ti metal coating can be applied by PVD, CVD, ALD and PE-CVD in an inert environment. The coating thickness of Ti metal is 0.5-15 microns. Thereafter, the Ti metal coating is exposed to nitrogen gas and/or a nitrogen containing gas compound to cause the nitrogen to react with the Ti metal coating to form a layer of TiN on the outer surface of the Ti metal coating and/or the outer surface of the metal alloy. In another non-limiting embodiment, the enhancement coating composition generally includes 20-85 wt. % Ti (and all values and ranges therebetween), 5-30 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement coating composition generally includes 70-80 wt. % Ti, 20-25 wt. % N, 0-8 wt. % Re, 0-1 wt. % Si, 0-1 wt. % O, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes a zirconium nitride (ZrN) coating. A portion or all of the outer surface of the metal alloy can include the zirconium nitride (ZrN) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZrN on the outer surface of the Zr metal coating and/or the outer surface of the metal alloy. The ZrN coating has been found to produce a gold-colored enhancement coating color. In another non-limiting embodiment, the enhancement coating composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement coating composition generally includes 80-90 wt. % Zr, 10-20 wt. % N, 0-8 wt. % Re, 0-1 wt. % Si, 0-1 wt. % O, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes a zirconium oxide (ZrO₂) coating. A portion or all of the outer surface of the metal alloy can include the zirconium oxide (ZrO₂) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO₂) on the outer surface of the Zr metal coating and/or the outer surface of the metal alloy. The zirconium oxide (ZrO₂) coating has been found to produce a blue colored enhancement coating color. In another non-limiting embodiment, the enhancement coating composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement coating composition generally includes 70-80 wt. % Zr, 20-30 wt. %, 0-1 wt. % N, 0-8 wt. % Re, 0-1 wt. % Si, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes both a zirconium oxide (ZrO₂) coating and a zirconium nitride coating (ZrN). A portion or all of the outer surface of the metal alloy can include the zirconium oxide (ZrO₂) coating and the zirconium nitride coating (ZrN). The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO₂) coating and the zirconium nitride coating (ZrN) are similar or the same as discussed above.

In accordance with another and/or alternative non-limiting aspect of the present disclosure, the metal alloy is coated with an enhancement coating to improve one or more properties of the metal alloy wherein the enhancement coating composition generally includes a zirconium oxycarbide (ZrOC) coating. A portion or all of the outer surface of the metal alloy can include the zirconium oxycarbide (ZrOC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. In another non-limiting embodiment, the enhancement coating composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-65 wt. % Zr, 5-25 wt. % O, and 25-40 wt. % C, 0-1 wt. % N, 0-8 wt. % Re, and 0-1 wt. % Si.

In accordance with another and/or alternative non-limiting aspect of the present disclosure, the metal alloy is coated with an enhancement coating to improve one or more properties of the metal alloy wherein the enhancement coating composition generally includes a zirconium-nitrogen-carbon (ZrNC) coating. A portion or all of the outer surface of the metal alloy can include the zirconium-nitrogen-carbon (ZrNC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZrNC will vary depending on the amount of C and N in the coating. In one non-limiting embodiment, the enhancement coating composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-80 wt. % Zr, 5-25 wt. % N, and 5-25 wt. % C, 0-1 wt. % O, 0-8 wt. % Re, and 0-1 wt. % Si.

In accordance with another and/or alternative non-limiting aspect of the present disclosure, the metal alloy that includes at least 5 atw. % rhenium and/or hafnium or a refractory metal alloy optionally has reduced ion release of the primary components of the metal alloy as compared to stainless steel, cobalt-chromium alloy, nickel-titanium alloy, or TiAlV alloy. In one non-limiting embodiment, and wherein the metal alloy that includes at least 5 atw. % rhenium and/or hafnium or refractory metal alloy has a maximum ion release of a primary component of the metal alloy when inserted or implanted on or in the body of the patient of no more than 0.5 μg/cm² per day (e.g., 0.001-0.5 μg/cm² per day and all values and ranges therebetween); and wherein the primary component is a component of the metal alloy that constitutes at least 2 wt. % of the metal alloy; and wherein the metal alloy optionally has an absolute increase in ion release per dose of metal alloy in the tissue about the implanted medical device of no more than 50 days (e.g., 5-50 days and all values and ranges therebetween) after inserted or implanted on or in the body of a patient; and wherein the metal alloy optionally has no more than 50% (e.g., 0-50% and all values and ranges therebetween) of the allowed daily exposure of primary metal form the metal alloy during the first 5 days after inserted or implanted on or in the body of a patient, and optionally has no more than 20% (e.g., 0-20% and all values and ranges therebetween) of the allowed daily exposure of primary metal form the metal alloy after the first 5 days after inserted or implanted on or in the body of a patient. As such, the metal alloy that includes at least 5 atw. % rhenium and/or hafnium or a refractory metal alloy optionally results in less potentially irritating metal ions (e.g., nickel ions, chromium ions, etc.) that are released from the metal alloy as compared to ion release from stainless steel, cobalt-chromium alloy, nickel-titanium alloy, or TiAlV alloy.

In accordance with another and/or alternative non-limiting aspect of the present disclosure, the metal alloy that includes at least 5 atw. % rhenium and/or hafnium or a refractory metal alloy optionally has increased hydrophilicity as compared to stainless steel, cobalt-chromium alloy, nickel-titanium alloy, or TiAlV alloy. Hydrophilicity of a material implanted in a patient is an important property of the material with regard to the cell adhesion, cell migration, and cell multiplication of tissue on the material. In one non-limiting embodiment, the metal alloy that includes at least 5 atw. % rhenium and/or hafnium or a refractory metal alloy optionally has a hydrophilicity wherein a contact angle of a water droplet on a surface of said metal alloy of 25-45° (e.g., 0.1-4.99 and all values and ranges therebetween). As a comparison, CoCr alloys are hydrophobic materials resulting in a large contact angle (93°±1) of a water droplet (e.g., distilled water) positioned on the surface of the CoCr alloy. TiAlV alloys are a little more hydrophilic than CoCr alloys and exhibit a contact angle of 58°±8° when a water droplet is positioned on the surface of the Ti alloy. Metal alloys such as a MoRe alloy have a much greater hydrophilicity and have a contact angle of 37°±3° when a water droplet is positioned on the surface of the MoRe alloy.

In accordance with another and/or alternative non-limiting aspect of the present disclosure, there is provided a non-limiting method for forming a portion or all of a medical device that includes the steps of a) providing metal alloy powder; and wherein the metal alloy power optionally has an average particle size of no more than 200 mesh (e.g., 200-635 mesh and all values and ranges therebetween; 20-74 microns and all values and ranges therebetween); and wherein the purity of the metal alloy power is optionally at least 90% (e.g., 90-100% purity and all values and ranges therebetween); b) consolidating the metal alloy powder into a general shape of a rod; and wherein the step of consolidating optionally includes subjecting the metal alloy powder to an isostatic process that optionally applies a uniform pressure of 50-700 MPa (and all values and ranges therebetween) from all sides on the metal powder; and wherein the step of consolidating optionally occurs in an inert atmosphere, an oxygen reducing atmosphere of hydrogen or an argon and hydrogen mixture, and/or under a vacuum; c) sintering the rod shaped consolidated metal alloy powder to form a rod green part; and wherein the rod green part optionally has an average density of 0.7-0.95 (and all values and ranges therebetween) of a minimum theoretical density; and wherein the rod green part optionally has an average density of 10-20 gm/cc (and all values and ranges therebetween); and wherein the consolidated metal alloy rod powder is optionally sintered at a temperature of 1000-3500° C. (and all values and ranges therebetween) to partially or fully fuse the metal alloy powder together to form the rod green part; and wherein the step of sintering optionally occurs in an inert atmosphere, an oxygen reducing atmosphere of hydrogen or an argon and hydrogen mixture, and/or under a vacuum; d) optionally subjecting the rod green part to a primary reduction process (e.g., swaging process, etc.) to reduce the original outer cross-sectional area of the rod green part to a first drawn down cross-sectional area by use of a reducing mechanism; the rod green part is drawn down one or more times in the reducing mechanism to a first drawn down cross-sectional area of the rod green part; and wherein the first drawn down cross-sectional area of the rod green part is optionally no more than 50% (e.g., 1-50% and all values and ranges therebetween) of the original outer cross-sectional area of the rod green part; and wherein the metal alloy rod after the primary secondary reduction process optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the step of optionally subjecting the rod green part to a primary reduction process optionally occurs in an inert atmosphere, an oxygen reducing atmosphere of hydrogen or an argon and hydrogen mixture, and/or under a vacuum; e) optionally subjecting the rod green part to one or more secondary reduction process to further reduce the outer cross-sectional area of the rod green part to a drawn down cross-sectional area that is less than the cross-sectional area obtained by the primary drawn down process by use of a reducing mechanism; the rod green part is drawn down one or more times in the reducing mechanism to a second, third fourth, etc. drawn down cross-sectional area of the rod green part; and wherein the second, third fourth, etc. drawn down cross-sectional area of the rod green part is optionally no more than 40% (e.g., 1-40% and all values and ranges therebetween) of the primary outer cross-sectional area of the rod green part or some subsequent drawn down outer cross-sectional area of the rod green part; and wherein the metal alloy rod after the final secondary reduction process optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the step of optionally subjecting the rod green part to one or more secondary reduction process optionally occurs in an inert atmosphere, an oxygen reducing atmosphere of hydrogen or an argon and hydrogen mixture, and/or under a vacuum; wherein the one or more secondary reduction processes optionally includes a swaging process; and wherein the swaging process optionally occurs at a temperature of 400° C.-1500° C. (and all values and ranges therebetween) in a controlled neutral or non-reducing environment (e.g., an inert atmosphere, an oxygen reducing atmosphere of hydrogen or an argon and hydrogen mixture, under a vacuum, etc.); f) optionally annealing the rod green part after the rod green part has obtained primary drawn down process and/or after the rod green part has obtained one or more secondary reduction process; and wherein the optional annealing step can optionally occur after certain drawing downs of the outer cross-sectional area of the rod green part, or after all but the last drawing down of the outer cross-sectional area of the rod green part, or after all of the drawing downs of the outer cross-sectional area of the rod green part; and wherein the step of annealing optionally includes controlling an atmosphere (e.g., an inert atmosphere, an oxygen reducing atmosphere of hydrogen or an argon and hydrogen mixture, under a vacuum, etc.) about the rod green part during the step of annealing so that the rod green part after the step of annealing optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the annealing temperature during the step of annealing is optionally greater than 500° C. (e.g. 500-1600° C. and all values and ranges therebetween); and wherein the optional step of annealing or the optional step of reduction of cross-sectional area forms a metal alloy rod form the rod green part; g) optionally subjecting the metal alloy rod to a gun drilling process and/or an EDM cutting process to form a metal alloy tube from the metal alloy rod; and wherein the optional step of gun drilling and/or EDM cutting optionally includes controlling an atmosphere (e.g., an inert atmosphere, an oxygen reducing atmosphere of hydrogen or an argon and hydrogen mixture, under a vacuum, etc.) about the metal alloy tube from the metal alloy rod during the step of gun drilling and/or EDM cutting; and h) optionally cutting, etching, grinding, laser cutting, and/or shaving the metal alloy rod or metal alloy tube to partially or fully form the medical device; and wherein the optional step of cutting, etching, grinding, laser cutting, and/or shaving the metal alloy rod or metal alloy tube optionally includes controlling an atmosphere (e.g., an inert atmosphere, an oxygen reducing atmosphere of hydrogen or an argon and hydrogen mixture, under a vacuum, etc.) about the metal rod during the step of cutting, etching, grinding, laser cutting, and/or shaving the metal alloy rod or metal alloy tube; and wherein the medical device is optionally selected from a rod, a stent, a medical device frame, a valve frame, or hypotube.

In accordance with another and/or alternative non-limiting aspect of the present disclosure, there is provided a non-limiting method step for forming a portion or all of a medical device wherein after the final optional secondary reduction process the metal alloy rod is not exposed to a heat treatment process that includes exposing the metal alloy rod a temperature of 500° C. or more.

In accordance with another and/or alternative non-limiting aspect of the present disclosure, there is provided a non-limiting method step for forming a portion or all of a medical device wherein one or more outer surfaces of the metal alloy are optionally subjected to a nitriding process a) prior to and/or after the primary reduction process, b) prior to and/or after the one or more optional secondary reduction processes, c) prior to and/or after the optional annealing process, d) prior to and/or after to the optionally subjecting the metal alloy rod to a gun drilling process and/or an EDM cutting process to form a metal alloy tube from the metal alloy rod, and/or e) prior to and/or after to the optionally cutting, etching, grinding, laser cutting, and/or shaving the metal alloy rod or metal alloy tube to partially or fully form the medical device.

In accordance with another and/or alternative non-limiting aspect of the present disclosure, there is provided a non-limiting method step for forming a portion or all of a medical device wherein one or more outer surfaces of the metal alloy are optionally coated with an agent, and optionally a polymer to optionally control the release rate of the agent.

In accordance with another and/or alternative non-limiting aspect of the present disclosure, there is provided a non-limiting method step for forming a portion or all of a medical device wherein one or more outer surfaces of the metal alloy are optionally coated with an enhancement coating.

In accordance with another and/or alternative non-limiting aspect of the present disclosure, there is provided a non-limiting method step for forming a portion or all of a medical device wherein the metal alloy optionally has reduced ion release of the primary components of the metal alloy as compared to stainless steel, cobalt-chromium alloy, nickel-titanium alloy, or TiAlV alloy; and wherein the metal alloy optionally has a maximum ion release of a primary component of the metal alloy when inserted or implanted on or in the body of the patient of no more than 0.5 μg/cm2 per day (e.g., 0.001-0.5 μg/cm2 per day and all values and ranges therebetween); and wherein the primary component is a component of the metal alloy that constitutes at least 2 wt. % of the metal alloy; and wherein the metal alloy optionally has an absolute increase in ion release per dose of metal alloy in the tissue about the implanted medical device of no more than 50 days (e.g., 5-50 days and all values and ranges therebetween) after inserted or implanted on or in the body of a patient; and wherein the metal alloy optionally has no more than 50% (e.g., 0-50% and all values and ranges therebetween) of the allowed daily exposure of primary metal form the metal alloy during the first 5 days after inserted or implanted on or in the body of a patient, and optionally has no more than 20% (e.g., 0-20% and all values and ranges therebetween) of the allowed daily exposure of primary metal form the metal alloy after the first 5 days after inserted or implanted on or in the body of a patient.

In accordance with another and/or alternative non-limiting aspect of the present disclosure, there is provided a non-limiting method step for forming a portion or all of a medical device wherein metal alloy optionally has increased hydrophilicity as compared to stainless steel, cobalt-chromium alloy, nickel-titanium alloy, or TiAlV alloy, and wherein the metal alloy optionally has a hydrophilicity wherein a contact angle of a water droplet on a surface of the metal alloy is 25-45° (e.g., 0.1-4.99 and all values and ranges therebetween).

In accordance with another and/or alternative non-limiting aspect of the present disclosure, there is provided a non-limiting method step for forming an expandable metal alloy tube after the metal alloy tube is cut (e.g., laser cut, etc.) to partially or fully form the configuration of the final shape of the metal alloy tube (e.g., stent, valve frame, hypotube, etc.), the cut metal alloy tube is optionally submitted to one or more of the following steps: a) heat treating the cut metal tube at a temperature of at least 500° C. (e.g., 500° C.-1500° C. and all values and ranges therebetween), and wherein the heat treatment optionally occurs in a non-oxidizing environment (e.g., hydrogen environment, argon environment, under a vacuum, an inert atmosphere, an argon and hydrogen mixture, etc.), b) deburr dross and other undesired material from the interior of the cavity in the cut metal alloy tube, c) electropolish an outer surface and/or inner cavity surface of the cut metal alloy tube, and/or d) expand the cut metal alloy tube to an expanded configuration or orientation and thereafter subject the expanded cut metal alloy tube to one or more of the following processing steps: I) heat treating the expanded cut metal tube at a temperature of at least 500° C. (e.g., 500° C.-1500° C. and all values and ranges therebetween), and wherein the heat treatment optionally occurs in a non-oxidizing environment (e.g., hydrogen environment, argon environment, under a vacuum, an inert atmosphere, an argon and hydrogen mixture, etc.), II) electropolish an outer surface and/or inner cavity surface of the expanded cut metal alloy tube, and/or III) coat the outer surface of the expanded cut metal alloy tube with an enhancement coating.

In accordance with another and/or alternative non-limiting aspect of the present disclosure, there is provided non-limiting method steps for forming a portion or all of a medical device that include:
a) providing metal powder that includes one or more metals selected from the group consisting of calcium, chromium, cobalt, copper, gold, hafnium, iron, lead, magnesium, molybdenum, nickel, niobium, osmium, platinum, rare earth metals, rhenium, silver, tantalum, technetium, titanium, tungsten, vanadium, yttrium, zinc, and zirconium. The average particle size of the metal powder optionally has a size than is less than 200 mesh (e.g., 200-635 mesh and all values and ranges therebetween and all values and ranges therebetween). The metal powder optionally has a purity of at least 90% (e.g., 90-100% purity and all values and ranges therebetween). The metal powder can be a pure metal powder and/or a metal alloy powder.
b) mixing the metal powder to provide a homogeneous and uniform mixture of the metal powder. The metal powder is sufficiently mixed so as to obtain a specific ratio of metals of the metal powder to obtain the intended metal alloy that is to be used to partially or fully form the medical device. The process for mixing the metal powder is non-limiting. The metal powder can optionally be mixed in an inert atmosphere, an oxygen reducing atmosphere of hydrogen or an argon and hydrogen mixture, and/or under a vacuum.
c) consolidating the homogeneous and uniform mixture of metal powders into a rod-shaped green part. The outer diameter and length of the rod-shaped green part is non-limiting. In one non-limiting configuration, the rod-shaped green part has a constant cross-sectional shape and constant cross-sectional area along 10-100% (and values and ranges therebetween) the longitudinal length of the rod-shaped green part. The step of consolidating optionally includes subjecting the homogeneous and uniform mixture of metal powder to an isostatic process. In one non-limiting configuration, the isostatic process optionally applies a uniform pressure of at least 70 MPa (e.g., 70-1000 MPa and all values and ranges therebetween) from one or more or all sides on the homogeneous and uniform mixture of metal powder to form the rod-shaped green part. The rod-shaped green part can have a variety of cross-sectional shapes (e.g., circular shaped, oval shaped, square shaped, rectangular shaped, triangular shaped, polygonal shapes, etc.). During the step of consolidation, the metal powder in the rod-shaped green part is typically not alloyed and/or sintered together. As such, the rod-shaped green part generally has a low structure integrity. The rod-shaped green part generally has a density of less than 80% of a density of a same metal alloy rod that is formed by casting. The consolidating of the metal powder to form the rod-shaped green part can optionally occur in an inert atmosphere, an oxygen reducing atmosphere of hydrogen or an argon and hydrogen mixture, and/or under a vacuum.
d) sintering the rod-shaped green part to fuse together a portion of all of the metal powder in said rod-shaped green part to form a semi-dense metal alloy rod having an original cross-sectional area. The density of the metal alloy rod is generally more dense than the rod-shaped green part (e.g., 5-200% more dense and all values and ranges therebetween). The metal alloy rod can optionally has a density of 40-98% of a density of a same metal alloy rod that is formed by casting after the step of sintering. The step of sintering generally occurs at a temperature of at least 800° C. (e.g., 800-3500° C. and all values and ranges therebetween) to partially or fully fuse together the metal powder in the rod-shaped green part to form the metal alloy rod. The metal alloy rod optionally has an average density of 5-20 gm/cc (e.g., and all values and ranges therebetween) after the step of sintering. The step of sintering can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the step of sintering.
e) subjecting the metal alloy rod to a primary reduction process to form a metal alloy rod that has a first drawn-down cross-sectional area that is less than the original outer cross-sectional area of the metal alloy rod after the sintering step. The primary reduction process optionally occurs at a temperature of 400-1900° C. (and all values and ranges therebetween). The metal alloy rod can be drawn down in cross-sectional area one or more times during the primary reduction process. In one non-limiting configuration, the metal alloy rod is continuously heated during the one or more times of drawing down during the primary reduction process. The first drawn down cross-sectional area optionally reduces the original outer cross-sectional area of the rod-shaped green part by no more than 50% (e.g., 5-50% and all values and ranges therebetween). The step of primary reduction process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the primary reduction process. The metal alloy rod after the primary reduction process has a density of greater than 80% (e.g., 80-95% and all values and ranges therebetween) of a density of a same metal alloy rod that is formed by casting. The primary reduction process optionally includes the use of a swagging process.
f) subjecting the metal alloy rod to one or more secondary process steps after the primary reduction process, wherein the one or more secondary process steps includes one or more steps selected from the group consisting of I) subjecting the metal alloy rod to a gun drilling process and/or an EDM cutting process to form a metal alloy tube from the metal alloy rod; II) cutting, etching, grinding, laser cutting, and/or shaving the metal alloy rod or the metal alloy tube to partially or fully form the medical device; III) subjecting the metal rod to a secondary reduction process and/or a final reduction process to reduce the cross-sectional area of the metal alloy rod to less than the first drawn down cross-sectional area; IV) subjecting one or more portions of an outer surface of the metal alloy rod or metal alloy tube to a nitriding process; V) coating one or more portions of an outer surface of the metal alloy rod or metal alloy tube with an agent and/or polymer; VI) coating one or more portions of an outer surface of the metal alloy rod or metal alloy tube with an enhancement coating, and/or VII) annealing the metal alloy rod after the primary reduction process and/or after the secondary reduction process.

In accordance with another and/or alternative non-limiting aspect of the present disclosure, the metal rod is optionally subjected to one or more annealing processes. The one or more annealing processes can occur after the primary reduction process and/or after the secondary reduction process. The step of annealing can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the step of annealing. The temperature during the step of annealing is optionally at least 500° C.

In accordance with another and/or alternative non-limiting aspect of the present disclosure, the metal rod is optionally subjected to a secondary reduction process to reduce the first drawn-down cross-sectional area to a second drawn-down cross-sectional area, and wherein the second drawn-down cross-sectional area is less than the first drawn-down cross-sectional area. The secondary reduction process generally occurs either a) after the metal alloy rod has been drawn down by 50% of the original outer cross-sectional area of the rod-shaped green part or b) after the first annealing of the metal alloy rod. The secondary reduction process optionally occurs at a temperature of 400-1900° C. (and all values and ranges therebetween). The metal alloy rod can be drawn down in cross-sectional area one or more times during the secondary primary reduction process. In one non-limiting configuration, the metal alloy rod is continuously heated during the one or more times of drawing down during the secondary reduction process. During each draw down during the secondary reduction process, the metal alloy rod is not reduced in cross-section by more than 40% (e.g., 2-40% and all values and ranges therebetween). During the secondary reduction process, the metal alloy rod can optionally be annealed one or more times. The secondary reduction process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the secondary reduction process. The metal alloy rod after the secondary reduction process has a density of greater than 90% (e.g., 90-99.99% and all values and ranges therebetween) of a density of a same metal alloy rod that is formed by casting. The metal alloy rod optionally has a higher density after the secondary reduction process than the density of the metal alloy rod after the primary reduction process. The secondary reduction process optionally includes the use of a swagging process.

In accordance with another and/or alternative non-limiting aspect of the present disclosure, the metal rod is optionally subjected to final reduction process to reduce cross-sectional area of the metal rod. The metal alloy rod after the final reduction process has a density of greater than 90% (e.g., 90-99.99% and all values and ranges therebetween) of a density of a same metal alloy rod that is formed by casting. The metal alloy rod optionally has a higher density after the final reduction process than the density of the metal alloy rod after the primary reduction process. The final reduction process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the final reduction process. The metal alloy rod after said final reduction process optionally has one or more of i) at least 125 Ksi yield strength, ii) at least 130 Ksi ultimate strength, iii) an elongation of at least 9%, and/or iv) a reduction in cross-sectional area from said original cross-sectional area of at least 40%. The final reduction process optionally includes the use of a swagging process.

In accordance with another and/or alternative non-limiting aspect of the present disclosure, the metal rod is optionally subjected to a gun drilling process and/or an EDM cutting process to form a metal alloy tube from the metal alloy rod. The gun drilling process and/or an EDM cutting process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the gun drilling process and/or an EDM cutting process.

In accordance with another and/or alternative non-limiting aspect of the present disclosure, the metal rod is optionally subjected to cutting, etching, grinding, laser cutting, and/or shaving the metal alloy rod or the metal alloy tube to partially or fully form the medical device. In one non-limiting embodiment, the medical device is selected from a stent, medical device frame, or hypotube.

In accordance with another and/or alternative non-limiting aspect of the present disclosure, the metal rod is optionally subjected to a nitriding process a) prior to and/or after the primary reduction process, b) prior to and/or after the secondary reduction process, c) prior to and/or after the final reduction process, d) prior to and/or after the annealing process, e) prior to and/or after the gun drilling process and/or said EDM cutting process, and/or f) prior to and/or after the cutting, etching, grinding, laser cutting, and/or shaving of the metal alloy rod or metal alloy tube.

In accordance with another and/or alternative non-limiting aspect of the present disclosure, one or more portions of an outer surface of the metal alloy rod or metal alloy tube is coated with an agent and/or polymer. In one non-limiting embodiment, there is provided a metal alloy tube that is configured to be expandable, and wherein the expandable metal alloy tube is expanded prior to coating the expandable metal alloy tube with the agent and/or polymer.

In accordance with another and/or alternative non-limiting aspect of the present disclosure, one or more portions of an outer surface of the metal alloy rod or metal alloy tube is coated with an enhancement coating. The enhancement coating optionally includes one or more coatings selected from the group consisting of chromium nitride (CrN), diamond-like carbon (DLC), titanium nitride (TiN), titanium oxynitride or titanium nitride oxide (TiNOx), zirconium nitride (ZrN), zirconium oxide (ZrO2), zirconium oxynitride (ZrNxOy), oxyzirconium-nitrogen-carbon (ZrNC), and/or zirconium OxyCarbide (ZrOC); said enhancement coating coated on said metal alloy rod or said metal alloy tube by one or more processes selected from the group consisting of physical vapor deposition (PVD) process, chemical vapor deposition (CVD) process, atomic layer deposition (ALD) process, or a plasma-enhanced chemical vapor deposition (PE-CVD) process. In one non-limiting embodiment, there is provided a metal alloy tube that is configured to be expandable, and wherein the expandable metal alloy tube is expanded prior to coating the expandable metal alloy tube with the enhancement coating.

In accordance with another and/or alternative non-limiting aspect of the present disclosure, the metal alloy that is used to partially or fully form the metal alloy rod or metal alloy tube has a) a maximum ion release of a primary component of the metal alloy when inserted or implanted on or in a body of a patient of no more than 0.5 μg/cm² per day (e.g., 0.0001-0.5 μg/cm² per day and all values and ranges therebetween); and wherein the primary component is a component of the metal alloy that constitutes at least 2 wt. % of the metal alloy; b) the metal alloy has an absolute increase in ion release per dose of the metal alloy in the tissue about the implanted medical device of no more than 50 days (e.g. 0.5-50 days and all values and ranges therebetween) after inserted or implanted on or in the body of the patient; c) the metal alloy has no more than 50% (e.g., 0-50% and all values and ranges therebetween) of the allowed daily exposure of primary metal from the metal alloy during the first 5 days after inserted or implanted on or in the body of the patient; and/or d) a hydrophilicity wherein a contact angle of a water droplet on a surface of the metal alloy is 25-45° (and all values and ranges therebetween).

In accordance with another and/or alternative non-limiting aspect of the present disclosure, the metal alloy is formed of I) at least 10-15 awt. % hafnium and/or rhenium and 50-78 wt. % iron, and one or more of a) 9-27 wt. % chromium, b) 0.1-26 wt. % nickel, c) 0.01-7 wt. % molybdenum, d) 0.01-16 wt. % manganese, e) 0.01-4 wt. % silicon, f) 0.01-2 wt. % titanium, g) 0.01-1 wt. % selenium, h) 0.01-1 wt. % niobium, i) 0.01-2 wt. % aluminum, j) 0.01-1 wt. % tantalum, k) 0.01-1 wt. % cobalt, l) 0.01-5 wt. % copper, m) 0.01-1 wt. % vanadium, and n) 0.01-2 wt. % tungsten; or II) at least 10-15 awt. % hafnium and/or rhenium and 35-68 wt. % cobalt, and one or more of a) 12-28 wt. % chromium, b) 0.01-38 wt. % nickel, c) 0.1-30 wt. % molybdenum, d) 0.01-2 wt. % manganese, e) 0.01-1 wt. % silicon, f) 0.01-18 wt. % tungsten, g) 0.01-0.5 wt. % lanthanum, h) 0.01-20 wt. % iron, i) 0.01-5 wt. % titanium, j) 0.01-2 wt. % niobium, k) 0.01-2 wt. % aluminum, l) 0.01-1 wt. % silicon, m) 0.01-0.5 wt. % boron, and n) 0.01-0.5 wt. % silver; or III) at least 10-15 awt. % hafnium and/or rhenium and 70-91.5 wt. % titanium, and one or more of a) 2-8 wt. % aluminum, b) 0.01-16 wt. % vanadium, c) 0.01-1 wt. % iron, d) 0.01-0.5 wt. % yttrium, e) 0.01-20 wt. % chromium, f) 0-16 wt. % molybdenum, g) 0.01-2 wt. % nickel, h) 0.01-12 wt. % tin, i) 0.01-6 wt. % zirconium, j) 0.01-2 wt. % tantalum, k) 0.01-4 wt. % niobium, l) 0.01-1 wt. % silicon, and m) 0.01-3 wt. % iron; or IV) at least 10-15 awt. % hafnium and/or rhenium, 35-84 wt. % tantalum, and one or more of a) 0.1-25 wt. % tungsten, b) 0.1-30 wt. % molybdenum, c) 0.01-45 wt. % niobium, d) 0.01-5 wt. % chromium, f) 0.01-5 wt. % titanium, g) 0.01-5 wt. % zirconium, and h) 0.01-4 wt. % hafnium; or V) at least 10-15 awt. % hafnium and/or rhenium, 40-85 wt. % niobium, and one or more of a) 0.01-20 wt. % molybdenum, b) 0.01-35 wt. % tantalum, c) 0.01-12 wt. % hafnium, d) 0.01-5 wt. % zirconium, e) 0.01-3 wt. % titanium, f) 0.01-15 wt. % tungsten, and g) 0.01-1 wt. % yttrium; or VI) at least 10-15 awt. % rhenium, 30-58 wt. % titanium, and 30-58 wt. % nickel; or VII) at least 10-15 awt. % hafnium and/or rhenium, and one or more of a) 1-85 awt. % chromium, b) 0.1-10 awt. % titanium, c) 0.1-10 awt. % molybdenum, and d) 0.1-10 awt. % zirconium; or VIII) at least 10-15 awt. % hafnium and/or rhenium, 15-32 wt. % chromium, 1-36% wt. % nickel, 2-18 wt. % molybdenum, 0-18 wt. % iron, 0-1 wt. % titanium, 0-0.15 wt. % manganese, 0-0.15 wt. % silver, 0-0.025 wt. % carbon, 0-16 wt. % tungsten, 0-2 wt. % Si, 0-2 wt. % aluminum, 0-1 wt. % iron, 30-68 wt. % cobalt; or IX) at least 10-15 awt. % hafnium and/or rhenium, 19-21 wt. % chromium, 34-36 wt. % nickel, 9-11 wt. % molybdenum, 1 wt. % max iron, 1 wt. % max titanium, 0.15 wt. % max manganese, 0.15 wt. % max silver, 0.025 wt. % max carbon, balance cobalt; or X) at least 10-15 awt. % hafnium and/or rhenium, 38-42 wt. % cobalt, 18-22 wt. % chromium, 14-18 wt. % iron, 13-17 wt. % nickel, 6-8 wt. % molybdenum; or XI) at least 10-15 awt. % hafnium and/or rhenium, 18-22 wt. % chromium, 14-16 wt. % tungsten, 9-11 wt. % nickel, balance cobalt; XII) at least 10-15 awt. % hafnium and/or rhenium, 5.5-6.75 wt. % aluminum, 3.5-4.5 wt. % vanadium, 85-93 wt. % titanium, 0-0.4 wt. % iron, 0-0.2 wt. % carbon; XIII) at least 10-15 awt. % hafnium and/or rhenium, 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium; XIV) at least 10-15 awt. % hafnium and/or rhenium, 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper; XV) at least 10-15 awt. % hafnium and/or rhenium, 30-98 wt. % nickel, 5-25 wt. % chromium, 0-65 wt. % iron, 0-30 wt. % molybdenum, 0-32% wt. % copper, 0-32% wt. % cobalt, 2-2 wt. % aluminum, 0-6 wt. % tantalum, 0-15% wt. % tungsten, 0-5 wt. % titanium, 0-6 wt. % niobium, 0-3 wt. % silicon; XVI) at least 10-15 awt. % hafnium and/or rhenium, 80-99 wt. % titanium, 0-6 wt. % aluminum, 0-3 wt. % tin, 0-1 wt. % palladium, 0-8 wt. % vanadium, 0-15% wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron; XVII) at least 10-15 awt. % hafnium and/or rhenium, 85-98 wt. % tungsten, 0-8 wt. % nickel, 0-5 wt. % copper, 0-5 wt. % molybdenum, 0-4 wt. % iron; XVIII) at least 10-15 awt. % hafnium and/or rhenium, 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum; XIX) at least 10-15 awt. % hafnium and/or rhenium, 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20% wt. % nickel; XX) at least 10-15 awt. % hafnium and/or rhenium, 32-38 wt. % nickel, 18-22 wt. % chromium, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. % phosphorous, 0-0.1 wt. % boron, 0-0.1 wt. % sulfur, and cobalt; XXI) at least 10-15 awt. % hafnium and/or rhenium, 95-98.5 wt. % copper, 1-4 wt. % beryllium, 0-1 wt. % cobalt, and 0-0.5 wt. % silicon; XXII) at least 10-15 awt. % hafnium and/or rhenium, 30-98 wt. % rhenium, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon; XXIII) at least 10-15 awt. % hafnium and/or rhenium, 42-58 wt. % nickel and 42-58 wt. % titanium; XXIV) at least 1 wt. % rhenium and/or hafnium and one or more metal alloying additives selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than rhenium, b) metals other than the one or more the metal alloying additives, c) carbon, d) oxygen, and e) nitrogen; XXV) at least 20 wt. % of primary metal and one or more metal alloying additives, and wherein the primary metal includes one or more metals selected from the group consisting of molybdenum, niobium, hafnium, rhenium, tantalum, and tungsten, and wherein the one or more metal alloying additives includes one or more metals selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than the primary metal, b) metals other than the one or more metal alloying additives, c) carbon, d) oxygen, and e) nitrogen; XXVI) stainless-steel; XXVII) CoCr alloy; XXVIII) TiAlV alloy; XXIX) aluminum alloy; XXX) nickel alloy; XXXI) titanium alloy; XXXII) tungsten alloy; XXXIII) molybdenum alloy; XXXIV) copper alloy; XXXV) beryllium-copper alloy; XXXVI) titanium-nickel alloy; XXXVII) refractory metal alloy; XXXVIII) metal alloy that is formed of stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) rhenium (e.g., 5-99 awt. % rhenium and all values and ranges therebetween); XXXIX) metal alloy that is formed of stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) hafnium (e.g., 5-99 awt. % hafnium and all values and ranges therebetween).

One non-limiting object of the present disclosure is the provision of metal alloy in accordance with the present disclosure that can be used to partially or fully form a medical device, automotive parts, springs, aerospace parts, industrial machinery and parts, tools (e.g., medical tools, industrial tools, household tools, etc.), etc.

Another and/or alternative non-limiting object of the present disclosure is the provision of a metal alloy in accordance with the present disclosure that can be coated partially or fully on an outer surface of a metal body (e.g., Ti or Ti alloys, Co or Co alloys, Cr alloys, CoCr alloys, stainless-steel, Fe and Fe alloys, Ni alloys, W alloys, Mo or Mo alloys, Re or Re alloys, Mo—Re alloys, Mo—Re—Cr alloys, Fe—Cr—Mo—CB alloys, dual phase austenitic steel, Hi-entropy alloy systems, WCu alloys, WRe alloys, etc.), polymer body, ceramic body, or composite body by a plasma coating process, a chemical vapor deposition process, or 3D printing process.

Another and/or alternative non-limiting object of the present disclosure is the provision of a cutting tool (e.g., metal cutting tool, etc.) that includes a metal body and an outer coating of metal alloy in accordance with the present disclosure that was coated by a plasma coating process, a chemical vapor deposition process, or 3D printing process.

Another and/or alternative non-limiting object of the present disclosure is the provision of a medical device or medical tool that includes a metal body, polymer body, ceramic body, or composite body and an outer coating of the metal alloy in accordance with the present disclosure that was coated by a plasma coating process, a chemical vapor deposition process, or 3D printing process.

Another and/or alternative non-limiting object of the present disclosure is the provision of a medical device that is partially or fully form of the metal alloy of the present disclosure and which medical device has improved procedural success rates.

Another and/or alternative non-limiting object of the present disclosure is the provision of a method and process for forming the metal alloy in accordance with the present disclosure that inhibits or prevents the formation of micro-cracks during the processing of the metal alloy.

Another and/or alternative non-limiting object of the present disclosure is the provision of a medical device or tool that is partially or fully formed of the metal alloy in accordance with the present disclosure and wherein the medical device or tool has improved physical properties.

Another and/or alternative non-limiting object of the present disclosure is the provision of a medical device or tool that is at least partially formed of the metal alloy in accordance with the present disclosure that has increased strength and/or hardness and can optionally also be used as a marker material.

Another and/or alternative non-limiting object of the present disclosure is the provision of a medical device or tool that at least partially includes the metal alloy in accordance with the present disclosure and which metal alloy enables the medical device or tool to be formed with less material without sacrificing the strength of the medical device or tool as compared to prior medical devices.

Another and/or alternative non-limiting object of the present disclosure is the provision of a medical device or tool that at least partially includes the metal alloy in accordance with the present disclosure that is simple and cost effective to manufacture.

Another and/or alternative non-limiting object of the present disclosure is the provision of a medical device or tool that at least partially includes the metal alloy in accordance with the present disclosure that is at least partially coated with one or more polymer coatings.

Another and/or alternative non-limiting object of the present disclosure is the provision of a medical device or tool that at least partially includes the metal alloy in accordance with the present disclosure that is coated with one or more agents.

Another and/or alternative non-limiting object of the present disclosure is the provision of a medical device or tool that at least partially includes the metal alloy in accordance with the present disclosure that has one or more polymer coatings to at least partially control the release rate of one or more agents.

Another and/or alternative non-limiting object of the present disclosure is the provision of a medical device or tool that at least partially includes the metal alloy in accordance with the present disclosure that includes one or more surface structures and/or micro-structures.

Another and/or alternative non-limiting object of the present disclosure is the provision of a method and process for forming the metal alloy in accordance with the present disclosure so as to partially or fully form a medical device or tool.

Another and/or alternative non-limiting object of the present disclosure is the provision of a medical device or tool that at least partially includes the metal alloy in accordance with the present disclosure that includes one or more markers.

Another and/or alternative non-limiting object of the present disclosure is the provision of a medical device or tool that at least partially includes the metal alloy in accordance with the present disclosure that includes and/or is used with one or more physical hindrances.

Another and/or alternative non-limiting object of the present disclosure is the provision of a medical device or tool that at least partially includes the metal alloy in accordance with the present disclosure that can be used in conjunction with one or more agents not on or in the medical device.

Another and/or alternative non-limiting object of the present disclosure is the provision of a method and process for forming the metal alloy in accordance with the present disclosure so as to inhibit or prevent the formation of micro-cracks during the processing of the metal alloy into a medical device or tool.

Another and/or alternative non-limiting object of the present disclosure is the provision of a method and process for forming the metal alloy in accordance with the present disclosure that inhibits or prevents the introduction of impurities into the metal alloy during the processing of the metal alloy into a medical device or tool.

Another and/or alternative non-limiting object of the present disclosure is the provision of a method and process for forming the metal alloy in accordance with the present disclosure that inhibits or prevents crack propagation and/or fatigue failure of the metal alloy.

Another and/or alternative non-limiting object of the present disclosure is the provision of a medical device that is used in orthopedics (e.g., orthopedic device, nail, rod, screw, post, cage, plate, pedicle screw, cap, hinge, joint system, wire, anchor, spacer, shaft, spinal implant, anchor, disk, ball, tension band, locking connector, bone implant, prosthetic implant or device to repair, replace and/or support a bone, etc.), which medical device may or may not be expandable, and which medical device is partially or fully formed of the metal alloy in accordance with the present disclosure.

Another and/or alternative non-limiting object of the present disclosure is the provision of a medical device that is in the form of implant for insertion into a body passageway (e.g., PFO device, stent, TAVR valve, valve, spinal implant, vascular implant; graft, guide wire, sheath, stent catheter, electrophysiology catheter, hypotube, catheter, etc.), which medical device may or may not be expandable, and which medical device is partially or fully formed of the metal alloy in accordance with the present disclosure.

Another and/or alternative non-limiting object of the present disclosure is the provision of a medical device that is used in dentistry and orthodontics (e.g., dental restorations, dental implants, crowns, bridges, braces, dentures, wire, anchors, spacers, retainers, tubes, pins, screws, posts, rods, plates, palatal expander, orthodontic headgear, orthodontic archwire, teeth aligners, quadhelix, etc.), which medical device may or may not be expandable, and which medical device is partially or fully formed of the metal alloy in accordance with the present disclosure.

Another and/or alternative non-limiting object of the present disclosure is the provision of a medical device in the form of a stent that can be used in spinal fusion applications, and which medical device is partially or fully formed of the metal alloy in accordance with the present disclosure.

Another and/or alternative non-limiting object of the present disclosure is the provision of a medical device that includes a metal alloy that has had a nitriding process to form a nitride layer on the outer surface of the metal alloy.

Another and/or alternative non-limiting object of the present disclosure is the provision of a medical device that includes a metal alloy that has had a swaging process.

Another and/or alternative non-limiting object of the present disclosure is the provision of a method for forming a medical device comprising the steps of a) forming a metal rod; the metal rod having a surface and an outer cross-sectional area; the metal rod is formed of a metal alloy; b) reducing the outer cross-sectional area of the metal rod to a first drawn down cross-sectional area by a reducing mechanism; the metal rod being drawn down at least once second drawn down cross-sectional area to obtain the first drawn down cross-sectional area; c) optionally annealing the metal rod prior to the metal rod having the outer cross-sectional area drawn down by more than about 50%; the optional annealing at a first annealing temperature in a low oxygen environment; the optional annealing occurring after the metal rod has been drawn down to the first drawn down cross-sectional area; d) optionally reducing the cross-sectional area of the metal rod to a second drawn down cross-sectional area by the reducing mechanism; the metal rod being drawn down at least once second drawn down cross-sectional area to obtain the second drawn down cross-sectional area; the second drawn down cross-sectional area smaller than the first drawn down cross-sectional area; the outer cross-sectional area of the metal rod reduced by no more than about 25% during each drawing down process second drawn down cross-sectional area; and, e) not annealing the metal rod after a final step of reducing the cross-sectional area of the metal rod.

Another and/or alternative non-limiting object of the present disclosure is the provision of a method for forming a medical device further including the step of hollowing out the metal rod to form a metal tube.

Another and/or alternative non-limiting object of the present disclosure is the provision of a method for forming a medical device wherein the step of optionally hollowing the metal rod to form the metal tube includes one or more of gun drilling, EDM cutting, and wire EDM cutting.

Another and/or alternative non-limiting object of the present disclosure is the provision of a method for forming a medical device further including the step of controlling an atmosphere about the metal rod during one or more of the steps of reducing and/or annealing so that the metal alloy of the metal rod after final step of reducing includes less than about 30 ppm nitrogen, less than about 150 ppm carbon, and less than about 100 ppm oxygen and a carbon to oxygen atomic ratio of at least about 0.2:1.

Another and/or alternative non-limiting object of the present disclosure is the provision of a method for forming a medical device wherein the step of forming the metal rod includes a process of isostatically pressing metal powder together and subsequently sintering the metal powder to form the metal rod; the process of isostatically pressing together the metal powder and/the process of sintering occurs in a controlled atmosphere; the metal rod has an average density of about 0.7-0.95 a minimum theoretical density of the metal alloy; the metal rod has an average density of at least 5 gm/cc; the controlled atmosphere including an inert atmosphere, an oxygen reducing atmosphere, or a partial or full vacuum.

Another and/or alternative non-limiting object of the present disclosure is the provision of a method for forming a medical device wherein the step of forming the metal rod includes a) partially or fully melting one or more metals or a master alloy, and b) extruding or casting the partially or fully melted one or more metals or a master alloy to form the metal rod.

Another and/or alternative non-limiting object of the present disclosure is the provision of a method for forming a medical device further including the step of nitriding the metal rod to form a nitride layer on the metal rod.

Another and/or alternative non-limiting object of the present disclosure is the provision of a method for forming a medical device wherein the step of annealing the metal rod includes the steps of a) annealing the metal rod at an annealing temperature of at least about 1480° C. for a time period of at least about 5 minutes when the metal rod has wall thickness of greater than about 0.015 inch, b) annealing the metal rod at an annealing temperature of about 1450-1480° C. for a time period of at least about 5 minutes when the metal rod has wall thickness of about 0.008-0.015 inch, and/or c) annealing the metal rod at an annealing temperature of less than about 1450° C. for a time period of at least about 5 minutes when the metal rod has wall thickness of less than about 0.008 inch.

Another and/or alternative non-limiting object of the present disclosure is the provision of a method for forming a medical device wherein the medical device is a stent, TAVR valve, orthopedic device, or spinal device.

Another and/or alternative non-limiting object of the present disclosure is the provision of a method for forming a medical device wherein the medical device is orthopedic rod, an expandable stent, and expandable valve, expandable graph, or expandable sheath.

Another and/or alternative non-limiting object of the present disclosure is the provision of a method for forming a medical device wherein the metal rod is cut to partially or fully form the medical device.

Another and/or alternative non-limiting object of the present disclosure is the provision of a method for forming a medical device that includes the steps of a) providing metal alloy powder; and wherein the metal alloy power optionally has an average particle size of less than 200 mesh (e.g., 200-635 mesh and all values and ranges therebetween; 20-74 microns and all values and ranges therebetween); and wherein the purity of the metal alloy power is optionally at least 90% (e.g., 90-100% purity and all values and ranges therebetween); b) consolidating the metal alloy powder into a general shape of a rod; and wherein the step of consolidating optionally includes subjecting the metal alloy powder to an isostatic process that optionally applies a uniform pressure of 50-700 MPa (and all values and ranges therebetween) from all sides on the metal powder; and wherein the step of consolidating optionally occurs in an inert atmosphere, an oxygen reducing atmosphere of hydrogen or an argon and hydrogen mixture, and/or under a vacuum; c) sintering the rod shaped consolidated metal alloy powder to form a rod green part; and wherein the rod green part optionally has an average density of 0.7-0.95 (and all values and ranges therebetween) of a minimum theoretical density; and wherein the rod green part optionally has an average density of 10-20 gm/cc (and all values and ranges therebetween); and wherein the consolidated metal alloy rod powder is optionally sintered at a temperature of 1000-3500° C. (and all values and ranges therebetween) to partially or fully fuse the metal alloy powder together to form the rod green part; and wherein the step of sintering optionally occurs in an inert atmosphere, an oxygen reducing atmosphere of hydrogen or an argon and hydrogen mixture, and/or under a vacuum; d) optionally subjecting the rod green part to a primary reduction process (e.g., swaging process, etc.) to reduce the original outer cross-sectional area of the rod green part to a first drawn down cross-sectional area by use of a reducing mechanism; the rod green part is drawn down one or more times in the reducing mechanism to a first drawn down cross-sectional area of the rod green part; and wherein the first drawn down cross-sectional area of the rod green part is optionally no more than 50% (e.g., 1-50% and all values and ranges therebetween) of the original outer cross-sectional area of the rod green part; and wherein the metal alloy rod after the primary secondary reduction process optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the step of optionally subjecting the rod green part to a primary reduction process optionally occurs in an inert atmosphere, an oxygen reducing atmosphere of hydrogen or an argon and hydrogen mixture, and/or under a vacuum; e) optionally subjecting the rod green part to one or more secondary reduction process to further reduce the outer cross-sectional area of the rod green part to a drawn down cross-sectional area that is less than the cross-sectional area obtained by the primary drawn down process by use of a reducing mechanism; the rod green part is drawn down one or more times in the reducing mechanism to a second, third fourth, etc. drawn down cross-sectional area of the rod green part; and wherein the second, third fourth, etc. drawn down cross-sectional area of the rod green part is optionally no more than 40% (e.g., 1-40% and all values and ranges therebetween) of the primary outer cross-sectional area of the rod green part or some subsequent drawn down outer cross-sectional area of the rod green part; and wherein the metal alloy rod after the final secondary reduction process optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the step of optionally subjecting the rod green part to one or more secondary reduction process optionally occurs in an inert atmosphere, an oxygen reducing atmosphere of hydrogen or an argon and hydrogen mixture, and/or under a vacuum; wherein the one or more secondary reduction processes optionally includes a swaging process; and wherein the swaging process optionally occurs at a temperature of 400° C.-1500° C. (and all values and ranges therebetween) in a controlled neutral or non-reducing environment (e.g., an inert atmosphere, an oxygen reducing atmosphere of hydrogen or an argon and hydrogen mixture, under a vacuum, etc.); f) optionally annealing the rod green part after the rod green part has obtained primary drawn down process and/or after the rod green part has obtained one or more secondary reduction process; and wherein the optional annealing step can optionally occur after certain drawing downs of the outer cross-sectional area of the rod green part, or after all but the last drawing down of the outer cross-sectional area of the rod green part, or after all of the drawing downs of the outer cross-sectional area of the rod green part; and wherein the step of annealing optionally includes controlling an atmosphere (e.g., an inert atmosphere, an oxygen reducing atmosphere of hydrogen or an argon and hydrogen mixture, under a vacuum, etc.) about the rod green part during the step of annealing so that the rod green part after the step of annealing optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the annealing temperature during the step of annealing is optionally greater than 500° C. (e.g. 500-1600° C. and all values and ranges therebetween); and wherein the optional step of annealing or the optional step of reduction of cross-sectional area forms a metal alloy rod form the rod green part; g) optionally subjecting the metal alloy rod to a gun drilling process and/or an EDM cutting process to form a metal alloy tube from the metal alloy rod; and wherein the optional step of gun drilling and/or EDM cutting optionally includes controlling an atmosphere (e.g., an inert atmosphere, an oxygen reducing atmosphere of hydrogen or an argon and hydrogen mixture, under a vacuum, etc.) about the metal alloy tube from the metal alloy rod during the step of gun drilling and/or EDM cutting; and h) optionally cutting, etching, grinding, laser cutting, and/or shaving the metal alloy rod or metal alloy tube to partially or fully form the medical device; and wherein the optional step of cutting, etching, grinding, laser cutting, and/or shaving the metal alloy rod or metal alloy tube optionally includes controlling an atmosphere (e.g., an inert atmosphere, an oxygen reducing atmosphere of hydrogen or an argon and hydrogen mixture, under a vacuum, etc.) about the metal rod during the step of cutting, etching, grinding, laser cutting, and/or shaving the metal alloy rod or metal alloy tube; and wherein the medical device is optionally selected from a rod, a stent, a medical device frame, a valve frame, or hypotube.

Another and/or alternative non-limiting object of the present disclosure is the provision of a method step for forming a portion or all of a medical device wherein after the final optional secondary reduction process the metal alloy rod is not exposed to a heat treatment process that includes exposing the metal alloy rod a temperature of 500° C. or more.

Another and/or alternative non-limiting object of the present disclosure is the provision of a method step for forming a portion or all of a medical device wherein one or more outer surfaces of the metal alloy are optionally subjected to a nitriding process a) prior to and/or after the primary reduction process, b) prior to and/or after the one or more optional secondary reduction processes, c) prior to and/or after the optional annealing process, d) prior to and/or after to the optionally subjecting the metal alloy rod to a gun drilling process and/or an EDM cutting process to form a metal alloy tube from the metal alloy rod, and/or e) prior to and/or after to the optionally cutting, etching, grinding, laser cutting, and/or shaving the metal alloy rod or metal alloy tube to partially or fully form the medical device.

Another and/or alternative non-limiting object of the present disclosure is the provision of a method step for forming a portion or all of a medical device wherein one or more outer surfaces of the metal alloy are optionally coated with an agent, and optionally a polymer to optionally control the release rate of the agent.

Another and/or alternative non-limiting object of the present disclosure is the provision of a medical device wherein a portion or all of a medical device wherein one or more outer surfaces of the metal alloy are optionally coated with an enhancement coating.

Another and/or alternative non-limiting object of the present disclosure is the provision of a medical device wherein the metal alloy optionally has reduced ion release of the primary components of the metal alloy as compared to stainless steel, cobalt-chromium alloy, nickel-titanium alloy, or TiAlV alloy; and wherein the metal alloy optionally has a maximum ion release of a primary component of the metal alloy when inserted or implanted on or in the body of the patient of no more than 0.5 μg/cm2 per day (e.g., 0.001-0.5 μg/cm2 per day and all values and ranges therebetween); and wherein the primary component is a component of the metal alloy that constitutes at least 2 wt. % of the metal alloy; and wherein the metal alloy optionally has an absolute increase in ion release per dose of metal alloy in the tissue about the implanted medical device of no more than 50 days (e.g., 5-50 days and all values and ranges therebetween) after inserted or implanted on or in the body of a patient; and wherein the metal alloy optionally has no more than 50% (e.g., 0-50% and all values and ranges therebetween) of the allowed daily exposure of primary metal form the metal alloy during the first 5 days after inserted or implanted on or in the body of a patient, and optionally has no more than 20% (e.g., 0-20% and all values and ranges therebetween) of the allowed daily exposure of primary metal form the metal alloy after the first 5 days after inserted or implanted on or in the body of a patient.

Another and/or alternative non-limiting object of the present disclosure is the provision of a medical device wherein the metal alloy optionally has increased hydrophilicity as compared to stainless steel, cobalt-chromium alloy, nickel-titanium alloy, or TiAlV alloy, and wherein the metal alloy optionally has a hydrophilicity wherein a contact angle of a water droplet on a surface of the metal alloy is 25-45° (e.g., 0.1-4.99 and all values and ranges therebetween).

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

Although example embodiments have been disclosed, a worker of ordinary skill in this art would recognize that certain modifications would come within the scope of the claims. For that reason, the following claims should be studied to determine their true scope and content.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure which, as a matter of language, might be said to fall therebetween. The disclosure has been described with reference to the preferred embodiments. These and other modifications of the preferred embodiments, as well as other embodiments of the disclosure, will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. A method for forming a medical device from a metal alloy rod; said method comprising the steps of:
a) providing metal alloy powder;
b. consolidating said metal alloy powder into a rod; said step of consolidating includes subjecting said metal alloy powder to an isostatic process that applies a uniform pressure of 400-700 MPa from all sides on said metal powder; said step of consolidating occurring in an inert atmosphere, an oxygen reducing atmosphere of hydrogen or an argon and hydrogen mixture, and/or under a vacuum;
c. sintering said rod of consolidated metal alloy powder to form a rod green part; said rod green part has an average density of 0.7-0.95 of a minimum theoretical density; said rod green part has an average density of 11-20 gm/cc; said consolidated metal alloy powder is sintered at a temperature of 1600-3500° C. to partially or fully fuse said metal alloy powder together to form said rod green part;
d. subjecting said rod green part to a primary reduction process to reduce said original outer cross-sectional area of said rod green part to a first drawn down cross-sectional area by use of a reducing mechanism; said rod green part is drawn down one or more times in said reducing mechanism to a first drawn down cross-sectional area of said rod green part; said first drawn down cross-sectional area of said rod green part is no more than 50% of said original outer cross-sectional area of said rod green part;
e. annealing said rod green part after said rod green part has obtained said first drawn down cross-sectional area; said step of annealing includes controlling an atmosphere about said rod green part during said step of annealing so that said rod green part after said step of annealing includes less than about 30 ppm nitrogen, less than about 150 ppm carbon, and less than about 100 ppm oxygen and a carbon to oxygen atomic ratio of at least about 0.2:1; an annealing temperature during said step of annealing is greater than 1200° C.;
f. subjecting said rod green part to one or more secondary reduction processes to form said metal alloy rod; said two or more secondary reduction processes cause a further reduction of said cross-sectional area of said rod green part from said first drawn down cross-sectional area to a second drawn down cross-sectional area by use of said reducing mechanism; each of said secondary reduction processes causing a reduction in said cross-sectional area of said rod green part by no more than 25%; said rod green part metal is annealed after each secondary reduction process except for a final secondary reduction process; after said final secondary reduction process said metal alloy rod is not exposed to a heat treatment process that includes exposing said metal alloy rod a temperature of 500° C. or more; and wherein said metal alloy rod after said final secondary reduction process includes less than about 30 ppm nitrogen, less than about 150 ppm carbon, and less than about 100 ppm oxygen and a carbon to oxygen atomic ratio of at least about 0.2:1;
g. subjecting said metal alloy rod to a gun drilling process and/or an EDM cutting process to form a metal alloy tube from said metal alloy rod; and
h. cutting, etching, grinding, laser cutting, and/or shaving said metal alloy tube to partially or fully form said medical device; said medical device is selected from a stent, medical device frame, or hypotube.

2. The method as defined in claim 1, wherein said two or more secondary reduction processes includes a swagging process; said swaging process occurs at a temperature of 400° C.-1500° C. in a controlled neutral or non-reducing environment.

3. A method for forming a medical device from a metal alloy rod; said method comprising the steps of:
a. providing metal alloy powder; said metal alloy power has an average particle size of less than 200 mesh; a purity of said metal alloy power is at least 90%
b. consolidating said metal alloy powder into a rod shaped consolidated metal alloy powder; said step of consolidating includes subjecting the metal alloy powder to an isostatic process that applies a uniform pressure of 50-700 MPa from all sides on said metal powder; said step of consolidating occurs in an inert atmosphere, an oxygen reducing atmosphere of hydrogen or an argon and hydrogen mixture, and/or under a vacuum;
c. sintering said rod shaped consolidated metal alloy powder to form a rod green part; said rod green part has an average density of 0.7-0.95 of a minimum theoretical density; said rod green part has an average density of 10-20 gm/cc; said consolidated metal alloy rod powder is sintered at a temperature of 1000-3500° C. to partially or fully fuse said metal alloy powder together to form said rod green part;
d. subjecting said rod green part to a primary reduction process to reduce an original outer cross-sectional area of said rod green part to a first drawn down cross-sectional area by use of a reducing mechanism; said first drawn down cross-sectional area is no more than 50% of an original outer cross-sectional area of said rod green part; said rod green part includes no more than 30 ppm nitrogen, no more than 150 ppm carbon, and no more than 100 ppm oxygen after said primary reduction process;
e. annealing said rod green part after said step of subjecting said rod green part to said primary reduction process so as to form a metal alloy rod; said step of annealing includes controlling an atmosphere about said rod green part during said step of annealing; said metal alloy rod after said step of annealing includes no more than 30 ppm nitrogen, no more than 150 ppm carbon, and no more than 100 ppm oxygen; an annealing temperature during said step of annealing is at least 500° C.; and
f. subjecting said metal rod to one or more secondary process steps selected from the group consisting of A) subjecting said metal alloy rod to a gun drilling process and/or an EDM cutting process to form a metal alloy tube from the metal alloy rod; B) cutting, etching, grinding, laser cutting, and/or shaving said metal alloy rod or said metal alloy tube to partially or fully form said medical device; c) subjecting said metal rod to one or more secondary reduction processes to further reduce an outer cross-sectional area of said metal alloy rod to a drawn down cross-sectional area that is less than said cross-sectional area of said rod green part by said primary drawn down process; d) subjecting one or more portions of an outer surface of said metal alloy rod or metal alloy tube to a nitriding process; e) coating one or more portions of an outer surface of said metal alloy rod or metal alloy tube with an agent and/or polymer; and/or f) coating one or more portions of an outer surface of said metal alloy rod or metal alloy tube with an enhancement coating.

4. The method as defined in claim 3, wherein said metal rod is subjected to said gun drilling process and/or said EDM cutting process to form said metal alloy tube.

5. The method as defined in claim 4, wherein said metal alloy tube is subjected to one or more of said cutting, etching, grinding, laser cutting, and/or shaving; said medical device is selected from a stent, a medical device frame, a valve frame, or hypotube.

6. The method as defined in claim 3, wherein said metal alloy rod or said metal alloy tube includes said enhancement coating; said enhancement coating includes one or more coatings selected from the group consisting of chromium nitride (CrN), diamond-like carbon (DLC), titanium nitride (TiN), titanium oxynitride or titanium nitride oxide (TiNOx), zirconium nitride (ZrN), zirconium oxide (ZrO2), zirconium oxynitride (ZrNxOy), oxyzirconium-nitrogen-carbon (ZrNC), and/or zirconium OxyCarbide (ZrOC); said enhancement coating coated on said metal alloy rod or said metal alloy tube by one or more processes selected from the group consisting of physical vapor deposition (PVD) process, chemical vapor deposition (CVD) process, atomic layer deposition (ALD) process, or a plasma-enhanced chemical vapor deposition (PE-CVD) process.

7. The method as defined in claim 3, wherein said metal alloy rod is subjected to one or more of said secondary reduction processes; each of said one or more of said secondary reduction processes drawing down said cross-sectional area of said metal rod by no more than 40%; said metal alloy rod after each of said secondary reduction processes includes no more than 30 ppm nitrogen, no more than 150 ppm carbon, and no more than 100 ppm oxygen; said secondary reduction processes includes a swaging process.

8. The method as defined in claim 7, wherein said metal alloy rod is not exposed to a heat treatment process that includes exposing the metal alloy rod a temperature of 500° C. or more after a final secondary reduction processes.

9. The method as defined in claim 3, wherein said metal alloy rod is subjected to said nitriding process a) prior to and/or after said primary reduction process, b) prior to and/or after said one or more optional secondary reduction processes, c) prior to and/or after said annealing process, d) prior to and/or after said gun drilling process and/or said EDM cutting process, or e) prior to and/or after said cutting, etching, grinding, laser cutting, and/or shaving of said metal alloy rod or metal alloy tube.

10. The method as defined in claim 3, wherein said metal alloy rod or metal alloy tube has a) a maximum ion release of a primary component of said metal alloy when inserted or implanted on or in a body of a patient of no more than 0.5 μg/cm² per day; and wherein said primary component is a component of said metal alloy that constitutes at least 2 wt. % of said metal alloy; b) said metal alloy has an absolute increase in ion release per dose of said metal alloy in the tissue about the implanted medical device of no more than 50 days after inserted or implanted on or in the body of the patient; c) said metal alloy has no more than 50% of the allowed daily exposure of primary metal from said metal alloy during the first 5 days after inserted or implanted on or in the body of the patient; and/or d) a hydrophilicity wherein a contact angle of a water droplet on a surface of said metal alloy is 25-45°.

11. The method as defined in claim 3, wherein said metal alloy is I) at least 10-15 awt. % hafnium and/or rhenium and 50-78 wt. % iron, and one or more of a) 9-27 wt. % chromium, b) 0.1-26 wt. % nickel, c) 0.01-7 wt. % molybdenum, d) 0.01-16 wt. % manganese, e) 0.01-4 wt. % silicon, f) 0.01-2 wt. % titanium, g) 0.01-1 wt. % selenium, h) 0.01-1 wt. % niobium, i) 0.01-2 wt. % aluminum, j) 0.01-1 wt. % tantalum, k) 0.01-1 wt. % cobalt, l) 0.01-5 wt. % copper, m) 0.01-1 wt. % vanadium, and n) 0.01-2 wt. % tungsten; or II) at least 10-15 awt. % hafnium and/or rhenium and 35-68 wt. % cobalt, and one or more of a) 12-28 wt. % chromium, b) 0.01-38 wt. % nickel, c) 0.1-30 wt. % molybdenum, d) 0.01-2 wt. % manganese, e) 0.01-1 wt. % silicon, f) 0.01-18 wt. % tungsten, g) 0.01-0.5 wt. % lanthanum, h) 0.01-20 wt. % iron, i) 0.01-5 wt. % titanium, j) 0.01-2 wt. % niobium, k) 0.01-2 wt. % aluminum, l) 0.01-1 wt. % silicon, m) 0.01-0.5 wt. % boron, and n) 0.01-0.5 wt. % silver; or III) at least 10-15 awt. % hafnium and/or rhenium and 70-91.5 wt. % titanium, and one or more of a) 2-8 wt. % aluminum, b) 0.01-16 wt. % vanadium, c) 0.01-1 wt. % iron, d) 0.01-0.5 wt. % yttrium, e) 0.01-20 wt. % chromium, f) 0-16 wt. % molybdenum, g) 0.01-2 wt. % nickel, h) 0.01-12 wt. % tin, i) 0.01-6 wt. % zirconium, j) 0.01-2 wt. % tantalum, k) 0.01-4 wt. % niobium, l) 0.01-1 wt. % silicon, and m) 0.01-3 wt. % iron; or IV) at least 10-15 awt. % hafnium and/or rhenium, 35-84 wt. % tantalum, and one or more of a) 0.1-25 wt. % tungsten, b) 0.1-30 wt. % molybdenum, c) 0.01-45 wt. % niobium, d) 0.01-5 wt. % chromium, f) 0.01-5 wt. % titanium, g) 0.01-5 wt. % zirconium, and h) 0.01-4 wt. % hafnium; or V) at least 10-15 awt. % hafnium and/or rhenium, 40-85 wt. % niobium, and one or more of a) 0.01-20 wt. % molybdenum, b) 0.01-35 wt. % tantalum, c) 0.01-12 wt. % hafnium, d) 0.01-5 wt. % zirconium, e) 0.01-3 wt. % titanium, f) 0.01-15 wt. % tungsten, and g) 0.01-1 wt. % yttrium; or VI) at least 10-15 awt. % rhenium, 30-58 wt. % titanium, and 30-58 wt. % nickel; or VII) at least 10-15 awt. % hafnium and/or rhenium, and one or more of a) 1-85 awt. % chromium, b) 0.1-10 awt. % titanium, c) 0.1-10 awt. % molybdenum, and d) 0.1-10 awt. % zirconium; or VIII) at least 10-15 awt. % hafnium and/or rhenium, 15-32 wt. % chromium, 1-36% wt. % nickel, 2-18 wt. % molybdenum, 0-18 wt. % iron, 0-1 wt. % titanium, 0-0.15 wt. % manganese, 0-0.15 wt. % silver, 0-0.025 wt. % carbon, 0-16 wt. % tungsten, 0-2 wt. % Si, 0-2 wt. % aluminum, 0-1 wt. % iron, 30-68 wt. % cobalt; or IX) at least 10-15 awt. % hafnium and/or rhenium, 19-21 wt. % chromium, 34-36 wt. % nickel, 9-11 wt. % molybdenum, 1 wt. % max iron, 1 wt. % max titanium, 0.15 wt. % max manganese, 0.15 wt. % max silver, 0.025 wt. % max carbon, balance cobalt; or X) at least 10-15 awt. % hafnium and/or rhenium, 38-42 wt. % cobalt, 18-22 wt. % chromium, 14-18 wt. % iron, 13-17 wt. % nickel, 6-8 wt. % molybdenum; or XI) at least 10-15 awt. % hafnium and/or rhenium, 18-22 wt. % chromium, 14-16 wt. % tungsten, 9-11 wt. % nickel, balance cobalt; XII) at least 10-15 awt. % hafnium and/or rhenium, 5.5-6.75 wt. % aluminum, 3.5-4.5 wt. % vanadium, 85-93 wt. % titanium, 0-0.4 wt. % iron, 0-0.2 wt. % carbon; XIII) at least 10-15 awt. % hafnium and/or rhenium, 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium; XIV) at least 10-15 awt. % hafnium and/or rhenium, 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper; XV) at least 10-15 awt. % hafnium and/or rhenium, 30-98 wt. % nickel, 5-25 wt. % chromium, 0-65 wt. % iron, 0-30 wt. % molybdenum, 0-32% wt. % copper, 0-32% wt. % cobalt, 2-2 wt. % aluminum, 0-6 wt. % tantalum, 0-15% wt. % tungsten, 0-5 wt. % titanium, 0-6 wt. % niobium, 0-3 wt. % silicon; XVI) at least 10-15 awt. % hafnium and/or rhenium, 80-99 wt. % titanium, 0-6 wt. % aluminum, 0-3 wt. % tin, 0-1 wt. % palladium, 0-8 wt. % vanadium, 0-15% wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron; XVII) at least 10-15 awt. % hafnium and/or rhenium, 85-98 wt. % tungsten, 0-8 wt. % nickel, 0-5 wt. % copper, 0-5 wt. % molybdenum, 0-4 wt. % iron; XVIII) at least 10-15 awt. % hafnium and/or rhenium, 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum; XIX) at least 10-15 awt. % hafnium and/or rhenium, 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20% wt. % nickel; XX) at least 10-15 awt. % hafnium and/or rhenium, 32-38 wt. % nickel, 18-22 wt. % chromium, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. % phosphorous, 0-0.1 wt. % boron, 0-0.1 wt. % sulfur, and cobalt; XXI) at least 10-15 awt. % hafnium and/or rhenium, 95-98.5 wt. % copper, 1-4 wt. % beryllium, 0-1 wt. % cobalt, and 0-0.5 wt. % silicon; XXII) at least 10-15 awt. % hafnium and/or rhenium, 30-98 wt. % rhenium, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon; XXIII) at least 10-15 awt. % hafnium and/or rhenium, 42-58 wt. % nickel and 42-58 wt. % titanium; XXIV) at least 1 wt. % rhenium and/or hafnium and one or more metal alloying additives selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than rhenium, b) metals other than the one or more the metal alloying additives, c) carbon, d) oxygen, and e) nitrogen; XXV) at least 20 wt. % of primary metal and one or more metal alloying additives, and wherein the primary metal includes one or more metals selected from the group consisting of molybdenum, niobium, hafnium, rhenium, tantalum, and tungsten, and wherein the one or more metal alloying additives includes one or more metals selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than the primary metal, b) metals other than the one or more metal alloying additives, c) carbon, d) oxygen, and e) nitrogen; XXVI) stainless-steel; XXVII) CoCr alloy; XXVIII) TiAlV alloy; XXIX) aluminum alloy; XXX) nickel alloy; XXXI) titanium alloy; XXXII) tungsten alloy; XXXIII) molybdenum alloy; XXXIV) copper alloy; XXXV) beryllium-copper alloy; XXXVI) titanium-nickel alloy; XXXVII) refractory metal alloy; XXXVIII) metal alloy that is formed of stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) rhenium (e.g., 5-99 awt. % rhenium and all values and ranges therebetween); XXXIX) metal alloy that is formed of stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) hafnium (e.g., 5-99 awt. % hafnium and all values and ranges therebetween).

12. A method for forming a medical device from a metal alloy; said method comprising the steps of:
a) providing metal powder containing one or more metals from the group consisting of calcium, chromium, cobalt, copper, gold, hafnium, iron, lead, magnesium, molybdenum, nickel, niobium, osmium, platinum, rare earth metals, rhenium, silver, tantalum, technetium, titanium, tungsten, vanadium, yttrium, zinc, and zirconium;
b) mixing said metal powder to provide a homogeneous and uniform mixture of said metal powders at a specific ratio for the intended metal alloy that is to be used to partially or fully form said medical device;
c) consolidating said homogeneous and uniform mixture of said metal powders into a rod-shaped green part; said step of consolidating includes subjecting said homogeneous and uniform mixture of said metal powders to an isostatic process that applies a uniform pressure of 200-1000 MPa from all sides on said homogeneous and uniform mixture of said metal powders to form said rod-shaped green part; said rod-shaped green part is a compacted metal powder part having a rod-shape, but said metal powder has not been alloyed and/or sintered, and wherein said rod-shaped green part has low structure integrity;
d) sintering said rod-shaped green part at a temperature of 1000-3500° C. to fuse together metal powder in said rod-shaped green part to form a semi-dense metal alloy rod having an original cross-sectional area;
e) subjecting said semi-dense metal alloy rod to a primary reduction process to form a metal alloy rod that has a first drawn-down cross-sectional area that is less than said original outer cross-sectional area of said semi-dense metal alloy rod; said primary reduction process occurs at a temperature of 400-1900° C.; said semi-dense metal alloy rod is drawn down in cross-sectional area one or more times during said primary reduction process; said semi-dense metal alloy rod is continually heated during said one or more times of drawing down during said primary reduction process;
f) subjecting said semi-dense metal alloy rod to a secondary reduction process to reduce said first drawn-down cross-sectional area to a second drawn-down cross-sectional area; said second drawn-down cross-sectional area is less than said first drawn-down cross-sectional area; said secondary reduction process occurs at a temperature of 400-1900° C.; said metal alloy rod is drawn down in cross-sectional area one or more times during said secondary reduction process; said metal alloy rod is continually heated during said one or more times of drawing down during said secondary reduction process;
g) subjecting said metal alloy rod to a final reduction process to reduce said second drawn-down cross-sectional area to a third drawn-down cross-sectional area; said third drawn-down cross-sectional area is less than said second drawn-down cross-sectional area; said secondary reduction process occurs at a temperature of less than 400° C.; said metal alloy rod after said final reduction process has a density of greater than 90% of a density of a same metal alloy rod that is formed by casting; said metal alloy rod after said final reduction process has one or more of i) at least 125 Ksi yield strength, ii) at least 130 Ksi ultimate strength, iii) an elongation of at least 9%, and/or iv) a reduction in cross-sectional area from said original cross-sectional area of at least 40%;
h) subjecting said metal alloy rod to a gun drilling process and/or an EDM cutting process to form a metal tube from said metal alloy rod; and
i) cutting, etching, grinding, laser cutting, and/or shaving said metal alloy tube to partially or fully form said medical device; said medical device is selected from a stent, medical device frame, or hypotube.

13. The method as defined in claim 12, wherein said metal powder is or includes an metal alloy powder.

14. The method as defined in claim 12, wherein one or more of said primary reduction process, said secondary reduction process and/or said final reduction process includes use of a swagging process; and wherein said swaging process used in said primary reduction process and/or said secondary reduction process occurs at a temperature of 400° C.-1500° C.; and wherein one or more of said primary reduction process, said secondary reduction process and/or said final reduction process occurs in a controlled neutral or non-reducing environment.

15. A method for forming a medical device from a metal rod; said method comprising the steps of:
a. providing metal powder; said metal powder has an average particle size of less than 200 mesh; a purity of said metal powder is at least 90%;
b. consolidating said metal powder to form a rod-shaped green part; said step of consolidating includes subjecting the metal powder to an isostatic process that applies a uniform pressure of 50-700 MPa from all sides on said metal powder; said step of consolidating occurs in an inert atmosphere, an oxygen reducing atmosphere of hydrogen or an argon and hydrogen mixture, and/or under a vacuum;
c. sintering said rod-shaped green part form a metal alloy rod; said metal alloy rod has said metal alloy rod after said final reduction process has a density of 70-98% of a density of a same metal alloy rod that is formed by casting; said metal alloy rod has an average density of 10-20 gm/cc; said step of sinter occurs at a temperature of 1000-3500° C. to partially or fully fuse said metal powder together to form said metal alloy rod;
d. subjecting said rod-shaped green part to a primary reduction process to reduce an original outer cross-sectional area of said rod-shaped green part to a first drawn down cross-sectional area by use of a reducing mechanism; said first drawn down cross-sectional area is no more than 50% of an original outer cross-sectional area of said rod-shaped green part; said metal alloy rod includes no more than 30 ppm nitrogen, no more than 150 ppm carbon, and no more than 100 ppm oxygen after said primary reduction process;
e. annealing said metal alloy rod after said primary reduction process; said step of annealing includes controlling an atmosphere about said rod-shaped green part during said step of annealing; said metal alloy rod after said step of annealing includes no more than 30 ppm nitrogen, no more than 150 ppm carbon, and no more than 100 ppm oxygen; an annealing temperature during said step of annealing is at least 500° C.; and
f. subjecting said metal rod to one or more secondary process steps selected from the group consisting of A) subjecting said metal alloy rod to a gun drilling process and/or an EDM cutting process to form a metal alloy tube from the metal alloy rod; B) cutting, etching, grinding, laser cutting, and/or shaving said metal alloy rod or said metal alloy tube to partially or fully form said medical device; c) subjecting said metal rod to a secondary reduction process and/or a final reduction process to reduce said cross-sectional area of said metal alloy rod to less than said first drawn down cross-sectional area; d) subjecting one or more portions of an outer surface of said metal alloy rod or metal alloy tube to a nitriding process; e) coating one or more portions of an outer surface of said metal alloy rod or metal alloy tube with an agent and/or polymer; and/or f) coating one or more portions of an outer surface of said metal alloy rod or metal alloy tube with an enhancement coating.

16. The method as defined in claim 15, wherein said metal rod is subjected to said gun drilling process and/or said EDM cutting process to form said metal alloy tube.

17. The method as defined in claim 15, wherein said metal alloy tube is subjected to one or more of said cutting, etching, grinding, laser cutting, and/or shaving; said medical device is selected from a stent, a medical device frame, a valve frame, or hypotube.

18. The method as defined in claim 15, wherein said metal alloy rod or said metal alloy tube includes said enhancement coating; said enhancement coating includes one or more coatings selected from the group consisting of chromium nitride (CrN), diamond-like carbon (DLC), titanium nitride (TiN), titanium oxynitride or titanium nitride oxide (TiNOx), zirconium nitride (ZrN), zirconium oxide (ZrO2), zirconium oxynitride (ZrNxOy), oxyzirconium-nitrogen-carbon (ZrNC), and/or zirconium OxyCarbide (ZrOC); said enhancement coating coated on said metal alloy rod or said metal alloy tube by one or more processes selected from the group consisting of physical vapor deposition (PVD) process, chemical vapor deposition (CVD) process, atomic layer deposition (ALD) process, or a plasma-enhanced chemical vapor deposition (PE-CVD) process.

19. The method as defined in claim 15, wherein said metal alloy rod is subjected to said secondary reduction process to reduce said metal alloy rod to a second drawn down cross-sectional area; said second drawn down cross-sectional area is less than said first drawn down cross-sectional area; during each of one or more drawing down process during said secondary reduction process a cross-sectional area of said metal alloy rod is reduced by no more than 40%; said metal alloy rod after said secondary reduction process includes no more than 30 ppm nitrogen, no more than 150 ppm carbon, and no more than 100 ppm oxygen; said secondary reduction process includes a swaging process.

20. The method as defined in claim 19, wherein said metal alloy rod is subjected to a final reduction process to reduce a cross-sectional area of said metal rod from said second drawn down cross-sectional area to a final drawn down cross-sectional area; said final drawn down cross-sectional area is less than said second drawn down cross-sectional area; said metal rod is exposed to a temperature of less than 500° C. during said final reduction process.

21. The method as defined in claim 15, wherein said metal alloy rod is subjected to said nitriding process a) prior to and/or after said primary reduction process, b) prior to and/or after said secondary reduction process, c) prior to and/or after said final reduction process, d) prior to and/or after said annealing process, e) prior to and/or after said gun drilling process and/or said EDM cutting process, and/or f) prior to and/or after said cutting, etching, grinding, laser cutting, and/or shaving of said metal alloy rod or metal alloy tube.

22. The method as defined in claim 15, wherein said metal alloy rod or metal alloy tube has a) a maximum ion release of a primary component of said metal alloy when inserted or implanted on or in a body of a patient of no more than 0.5 μg/cm² per day; and wherein said primary component is a component of said metal alloy that constitutes at least 2 wt. % of said metal alloy; b) said metal alloy has an absolute increase in ion release per dose of said metal alloy in the tissue about the implanted medical device of no more than 50 days after inserted or implanted on or in the body of the patient; c) said metal alloy has no more than 50% of the allowed daily exposure of primary metal from said metal alloy during the first 5 days after inserted or implanted on or in the body of the patient; and/or d) a hydrophilicity wherein a contact angle of a water droplet on a surface of said metal alloy is 25-45°.

23. The method as defined in claim 15, wherein said metal alloy is I) at least 10-15 awt. % hafnium and/or rhenium and 50-78 wt. % iron, and one or more of a) 9-27 wt. % chromium, b) 0.1-26 wt. % nickel, c) 0.01-7 wt. % molybdenum, d) 0.01-16 wt. % manganese, e) 0.01-4 wt. % silicon, f) 0.01-2 wt. % titanium, g) 0.01-1 wt. % selenium, h) 0.01-1 wt. % niobium, i) 0.01-2 wt. % aluminum, j) 0.01-1 wt. % tantalum, k) 0.01-1 wt. % cobalt, l) 0.01-5 wt. % copper, m) 0.01-1 wt. % vanadium, and n) 0.01-2 wt. % tungsten; or II) at least 10-15 awt. % hafnium and/or rhenium and 35-68 wt. % cobalt, and one or more of a) 12-28 wt. % chromium, b) 0.01-38 wt. % nickel, c) 0.1-30 wt. % molybdenum, d) 0.01-2 wt. % manganese, e) 0.01-1 wt. % silicon, f) 0.01-18 wt. % tungsten, g) 0.01-0.5 wt. % lanthanum, h) 0.01-20 wt. % iron, i) 0.01-5 wt. % titanium, j) 0.01-2 wt. % niobium, k) 0.01-2 wt. % aluminum, l) 0.01-1 wt. % silicon, m) 0.01-0.5 wt. % boron, and n) 0.01-0.5 wt. % silver; or III) at least 10-15 awt. % hafnium and/or rhenium and 70-91.5 wt. % titanium, and one or more of a) 2-8 wt. % aluminum, b) 0.01-16 wt. % vanadium, c) 0.01-1 wt. % iron, d) 0.01-0.5 wt. % yttrium, e) 0.01-20 wt. % chromium, f) 0-16 wt. % molybdenum, g) 0.01-2 wt. % nickel, h) 0.01-12 wt. % tin, i) 0.01-6 wt. % zirconium, j) 0.01-2 wt. % tantalum, k) 0.01-4 wt. % niobium, l) 0.01-1 wt. % silicon, and m) 0.01-3 wt. % iron; or IV) at least 10-15 awt. % hafnium and/or rhenium, 35-84 wt. % tantalum, and one or more of a) 0.1-25 wt. % tungsten, b) 0.1-30 wt. % molybdenum, c) 0.01-45 wt. % niobium, d) 0.01-5 wt. % chromium, f) 0.01-5 wt. % titanium, g) 0.01-5 wt. % zirconium, and h) 0.01-4 wt. % hafnium; or V) at least 10-15 awt. % hafnium and/or rhenium, 40-85 wt. % niobium, and one or more of a) 0.01-20 wt. % molybdenum, b) 0.01-35 wt. % tantalum, c) 0.01-12 wt. % hafnium, d) 0.01-5 wt. % zirconium, e) 0.01-3 wt. % titanium, f) 0.01-15 wt. % tungsten, and g) 0.01-1 wt. % yttrium; or VI) at least 10-15 awt. % rhenium, 30-58 wt. % titanium, and 30-58 wt. % nickel; or VII) at least 10-15 awt. % hafnium and/or rhenium, and one or more of a) 1-85 awt. % chromium, b) 0.1-10 awt. % titanium, c) 0.1-10 awt. % molybdenum, and d) 0.1-10 awt. % zirconium; or VIII) at least 10-15 awt. % hafnium and/or rhenium, 15-32 wt. % chromium, 1-36% wt. % nickel, 2-18 wt. % molybdenum, 0-18 wt. % iron, 0-1 wt. % titanium, 0-0.15 wt. % manganese, 0-0.15 wt. % silver, 0-0.025 wt. % carbon, 0-16 wt. % tungsten, 0-2 wt. % Si, 0-2 wt. % aluminum, 0-1 wt. % iron, 30-68 wt. % cobalt; or IX) at least 10-15 awt. % hafnium and/or rhenium, 19-21 wt. % chromium, 34-36 wt. % nickel, 9-11 wt. % molybdenum, 1 wt. % max iron, 1 wt. % max titanium, 0.15 wt. % max manganese, 0.15 wt. % max silver, 0.025 wt. % max carbon, balance cobalt; or X) at least 10-15 awt. % hafnium and/or rhenium, 38-42 wt. % cobalt, 18-22 wt. % chromium, 14-18 wt. % iron, 13-17 wt. % nickel, 6-8 wt. % molybdenum; or XI) at least 10-15 awt. % hafnium and/or rhenium, 18-22 wt. % chromium, 14-16 wt. % tungsten, 9-11 wt. % nickel, balance cobalt; XII) at least 10-15 awt. % hafnium and/or rhenium, 5.5-6.75 wt. % aluminum, 3.5-4.5 wt. % vanadium, 85-93 wt. % titanium, 0-0.4 wt. % iron, 0-0.2 wt. % carbon; XIII) at least 10-15 awt. % hafnium and/or rhenium, 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium; XIV) at least 10-15 awt. % hafnium and/or rhenium, 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper; XV) at least 10-15 awt. % hafnium and/or rhenium, 30-98 wt. % nickel, 5-25 wt. % chromium, 0-65 wt. % iron, 0-30 wt. % molybdenum, 0-32% wt. % copper, 0-32% wt. % cobalt, 2-2 wt. % aluminum, 0-6 wt. % tantalum, 0-15% wt. % tungsten, 0-5 wt. % titanium, 0-6 wt. % niobium, 0-3 wt. % silicon; XVI) at least 10-15 awt. % hafnium and/or rhenium, 80-99 wt. % titanium, 0-6 wt. % aluminum, 0-3 wt. % tin, 0-1 wt. % palladium, 0-8 wt. % vanadium, 0-15% wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron; XVII) at least 10-15 awt. % hafnium and/or rhenium, 85-98 wt. % tungsten, 0-8 wt. % nickel, 0-5 wt. % copper, 0-5 wt. % molybdenum, 0-4 wt. % iron; XVIII) at least 10-15 awt. % hafnium and/or rhenium, 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum; XIX) at least 10-15 awt. % hafnium and/or rhenium, 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20% wt. % nickel; XX) at least 10-15 awt. % hafnium and/or rhenium, 32-38 wt. % nickel, 18-22 wt. % chromium, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. % phosphorous, 0-0.1 wt. % boron, 0-0.1 wt. % sulfur, and cobalt; XXI) at least 10-15 awt. % hafnium and/or rhenium, 95-98.5 wt. % copper, 1-4 wt. % beryllium, 0-1 wt. % cobalt, and 0-0.5 wt. % silicon; XXII) at least 10-15 awt. % hafnium and/or rhenium, 30-98 wt. % rhenium, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon; XXIII) at least 10-15 awt. % hafnium and/or rhenium, 42-58 wt. % nickel and 42-58 wt. % titanium; XXIV) at least 1 wt. % rhenium and/or hafnium and one or more metal alloying additives selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than rhenium, b) metals other than the one or more the metal alloying additives, c) carbon, d) oxygen, and e) nitrogen; XXV) at least 20 wt. % of primary metal and one or more metal alloying additives, and wherein the primary metal includes one or more metals selected from the group consisting of molybdenum, niobium, hafnium, rhenium, tantalum, and tungsten, and wherein the one or more metal alloying additives includes one or more metals selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than the primary metal, b) metals other than the one or more metal alloying additives, c) carbon, d) oxygen, and e) nitrogen; XXVI) stainless-steel; XXVII) CoCr alloy; XXVIII) TiAlV alloy; XXIX) aluminum alloy; XXX) nickel alloy; XXXI) titanium alloy; XXXII) tungsten alloy; XXXIII) molybdenum alloy; XXXIV) copper alloy; XXXV) beryllium-copper alloy; XXXVI) titanium-nickel alloy; XXXVII) refractory metal alloy; XXXVIII) metal alloy that is formed of stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) rhenium (e.g., 5-99 awt. % rhenium and all values and ranges therebetween); XXXIX) metal alloy that is formed of stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) hafnium (e.g., 5-99 awt. % hafnium and all values and ranges therebetween).
